(19) United States
(12) Patent Application Publication
(10) Pub. No.: US 2023/0313189 A1
(43) Pub. Date: Oct. 5, 2023

(54) COMPLEMENT COMPONENT 4 INHIBITORS FOR TREATING NEUROLOGICAL DISEASES, AND RELATED COMPOSITONS, SYSTEMS AND METHODS OF USING SAME

(71) Applicant: Genentech, Inc., South San Francisco, CA (US)

(72) Inventors: Alexander MUNK, Ishoj (DK); Helene M. GYLLING, Copenhagen (DK); Jesse Eric HANSON, Belmont, CA (US); Lukasz J. KIELPINSKI, Hørsholm (DK)

(73) Assignee: Genentech, Inc., South San Francisco, CA (US)

(21) Appl. No.: 18/054,116

(22) Filed: Nov. 9, 2022

Related U.S. Application Data
(63) Continuation of application No. PCT/US2021/031265, filed on May 7, 2021.
(60) Provisional application No. 63/023,103, filed on May 11, 2020.

(51) Int. Cl. C12N 15/113; C12Q 1/6883

(52) U.S. Cl. CPC C12N 15/113; C12Q 1/6883; C12N 2310/11; C12N 2310/3231; C12N 2310/315; C12N 2310/341; C12N 2310/3341

(57) ABSTRACT

The present invention relates to complement component 4 (C4) inhibitors for use in treatment of a neurological disease. The invention in particular relates to the use of C4 inhibitors for down-regulation of C4 expression. The invention also relates to nucleic acid molecules, which are complementary to C4A and/or C4B and capable of reducing the level of an C4A and/or C4B mRNA. Also comprised in the present invention is a pharmaceutical composition and its use in the treatment of a neurological disease.

### CROSS-REFERENCE TO RELATED APPLICATIONS

This application relates to U.S. Provisional application filed May 11, 2020, entitled “Complement Component C1R Inhibitors For Treating A Neurological Disease, And Related Compositions, Systems And Methods Of Using Same” and US Provisional application filed May 11, 2020, entitled “Complement Component C1S Inhibitors For Treating A Neurological Disease, And Related Compositions, Systems And Methods Of Using Same,” the contents of which are both incorporated herein by reference in their entireties. This application claims priority to U.S. Provisional Application No. 63/023,103, filed May 11, 2020, entitled “Complement Component C4 Inhibitors For Treating A Neurological Disease, And Related Compositions, Systems And Methods Of Using Same,” the contents of which are incorporated herein by reference in its entirety.

### SEQUENCE LISTING

This application contains a Sequence Listing, which has been submitted electronically in XML format and is hereby incorporated by reference in its entirety. Said XML copy, created on May 3, 2023, is named P36089-US-1_SL.xml and is 3,268,941 bytes in size.

### FIELD OF INVENTION

The present invention relates to complement component 4 (C4) inhibitors for use in treatment of neurological diseases. The invention in particular relates to the use of C4 inhibitors for down-regulation of C4 expression. The invention also relates to nucleic acid molecules, which are complementary to C4A and/or C4B and capable of reducing the level of an C4A and/or C4B mRNA. Also comprised in the present invention is a pharmaceutical composition and its use in the treatment of neurological diseases.

### BACKGROUND

The complement system is a part of the innate immune system that enhances the clearance of microbes or damaged cells by phagocytes and promotes inflammation. The complement system also participates in synaptic pruning in the brain, with the classical pathway of the complement system mediating synapse removal. This process involves initiation of the classical pathway by the complement component 1 (C0) complex (consisting of C1Q, C1S and C1R), leading to cleavage of complement component 2 (C2) and complement component 4 (C4), which in turn lead to cleavage of complement component 3 (C3) followed by engulfment of synapses by microglia cells, Beyond roles in normal brain circuitry refinement during early development, it is well established that aberrant activity of the classical complement pathway can mediate synapse loss and neurodegeneration in various neurological diseases. Observations of elevated complement levels in patient samples and beneficial effects of reducing or eliminating complement components in mouse models have identified a damaging role for complement in conditions including, Alzheimer's disease, frontotemporal dementia, multiple sclerosis, amyotrophic lateral sclerosis, Huntington's disease, Parkinson's disease, virus-induced cognitive impairment, glaucoma, macular degeneration, myasthenia gravis, Guillain-Barré syndrome, neuromyelitis optica, central nervous system lupus erythematosus and schizophrenia.

There remains a need in the art for therapeutic and prognostic agents to address such conditions. The present invention meets these and other needs.

### OBJECTIVE OF THE INVENTION

The present invention provides nucleic acid inhibitors of complement component 4 (C4) which may be used both in vivo and in vitro for down-regulation of C4 expression and for the prophylactic and therapeutic intervention in neurological diseases. The present invention further identifies novel nucleic acid molecules, such as antisense oligonucleotides, which are capable of inhibiting the expression of C4 in vitro and in vivo.

### SUMMARY OF INVENTION

The present invention relates to oligonucleotides targeting a nucleic acid and capable of modulating the expression of C4, useful, for example, to treat or prevent diseases related to the functioning of C4.

Accordingly, in a first aspect, the invention provides a C4 inhibitor for use in the treatment and/or prevention of neurological diseases, such as tauopathies or schizophrenia, in particular, a C4 inhibitor is capable of reducing the amount of C4, such as C4 mRNA and/or C4 protein. Such an inhibitor is advantageously a nucleic acid molecule of 12 to 60 nucleotides in length, which is capable of reducing C4 mRNA levels. In some embodiments, C4 is C4A and/or C4B.

In a further aspect, the invention relates to a nucleic acid molecule of 12-60 nucleotides, such as of 12-30 nucleotides, comprising a contiguous nucleotide sequence of at least 10 nucleotides, in particular of 16 to 20 nucleotides, which is at least 90% complementary, such as 90-95%, 95-98%, or fully complementary to a mammalian C4, e.g. a human C4A and/or C4B, a mouse C4b and/or C4a or a cynomolgus monkey C4. Such a nucleic acid molecule is capable of inhibiting the expression of C4A and/or C4B in a cell expressing C4A and/or C4B. The inhibition of C4A and/or C4B expression allows for a reduction of the amount of C4A and/or C4B protein present in the cell. The nucleic acid molecule can be selected from a single stranded antisense oligonucleotide, a double stranded siRNA molecule or a shRNA nucleic acid molecule (in particular chemically produced shRNA molecules).

A further aspect of the present invention relates to single stranded antisense oligonucleotides or siRNAs that inhibit the expression and/or activity of C4A and/or C4B. In particular, modified antisense oligonucleotides or modified siRNAs comprising one or more 2′ sugar modified nucleoside(s) and one or more phosphorothioate linkage(s), which reduce C4A and/or C4B mRNA are advantageous.

In a further aspect, the invention provides pharmaceutical compositions comprising the C4 inhibitor of the present invention, such as the antisense oligonucleotide or siRNA of the invention and a pharmaceutically acceptable excipient.

In some embodiments, the C4 inhibitor is selected from the group consisting of a C4A inhibitor, a C4B inhibitor and a pan-C4 inhibitor.

In a further aspect, the invention provides methods for in vivo or in vitro modulation of C4A and/or C4B expression in a target cell, which is expressing C4A and/or C4B, by administering a C4 inhibitor of the present invention, such as an antisense oligonucleotide or composition of the invention in an effective amount to said cell. In some embodiments, the C4A and/or C4B expression is reduced by at least 50%, e.g., 50-60%; or at least 60%, e.g., 60-70%; or at least 70%, e.g., 70-80%; or at least 80%, e.g., 80-90%; or at least 90%, e.g., 90-95%, in the target cell compared to the level without any treatment or treated with a control.

In a further aspect, the invention provides methods for treating or preventing a disease, disorder or dysfunction associated with in vivo activity of C4 comprising administering a therapeutically or prophylactically effective amount of the C4 inhibitor of the present invention, such as the antisense oligonucleotide or siRNA of the invention to a subject suffering from or susceptible to the disease, disorder or dysfunction.

In some embodiments, the C4 inhibitor is selected from the group consisting of a C4A inhibitor, a C4B inhibitor and a pan-C4 inhibitor.

### Definitions

Compound

Herein, the term “compound”, with respect to a compound of the invention, means any molecule capable of inhibition C4 expression or activity. Particular compounds of the invention are nucleic acid molecules, such as RNAi molecules or antisense oligonucleotides according to the invention or any conjugate comprising such a nucleic acid molecule. For example, herein the compound may be a nucleic acid molecule targeting C4A and/or C4B, in particular an antisense oligonucleotide or a siRNA. In some embodiments, the compound is herein also referred to as an “inhibitor” or a “C4 inhibitor”. In some embodiments, the C4 inhibitor is selected from the group consisting of a C4A inhibitor, a C4B inhibitor and a pan-C4 inhibitor. The term “C4A inhibitor” as used herein designates a molecule capable of specifically inhibiting C4A expression or activity. The term “C4B inhibitor” as used herein designates a molecule capable of specifically inhibiting C4B expression or activity. The term “pan-C4 inhibitor” as used herein designates a molecule capable of inhibiting both, C4A and C4B expression or activity.

Oligonucleotide

The term “oligonucleotide” as used herein is defined as it is generally understood by the skilled person, such as, as a molecule comprising two or more covalently linked nucleosides. An oligonucleotide is also referred to herein as a “nucleic acid” or “nucleic acid molecule”. Such covalently bound nucleosides may also be referred to as nucleic acid molecules or oligomers. The oligonucleotides referred to in the description and claims are generally therapeutic oligonucleotides below 70 nucleotides in length. The oligonucleotide may be or comprise a single stranded antisense oligonucleotide, or may be another nucleic acid molecule, such as a CRISPR RNA, an siRNA, an shRNA, an aptamer, or a ribozyme. Therapeutic oligonucleotide molecules are commonly made in the laboratory by solid-phase chemical synthesis followed by purification and isolation. shRNA's are often delivered to cells using lentiviral vectors from which they are then transcribed to produce single stranded RNA that will form a stem loop (hairpin) RNA structure capable of interacting with RNA interference machinery (including the RNA-induced silencing complex (RISC)). In an embodiment of the present invention, the shRNA is a chemically produced shRNA molecule (not relying on cell-based expression from plasmids or viruses). When referring to a sequence of the oligonucleotide, reference is made to the sequence or order of nucleobase moieties, or modifications thereof, of the covalently linked nucleotides or nucleosides. Generally, the oligonucleotide of the invention is man-made, and is chemically synthesized, and is typically purified or isolated. Although in some embodiments, the oligonucleotide of the invention is an shRNA transcribed from a vector upon entry into the target cell. The oligonucleotide of the invention may comprise one or more modified nucleosides or nucleotides.

In some embodiments, the term oligonucleotide of the invention also includes pharmaceutically acceptable salts, esters, solvates and prodrugs thereof.

In some embodiments, the oligonucleotide of the invention comprises or consists of 10 to 70 nucleotides in length, such as from 12 to 60, such as from 13 to 50, such as from 14 to 40, such as from 15 to 30, such as from 16 to 25, such as from 16 to 22, such as from 16 to 20 contiguous nucleotides in length. Accordingly, the oligonucleotide of the present invention, in some embodiments, may have a length of 12 to 25 nucleotides. Alternatively, the oligonucleotide of the present invention, in some embodiments, may have a length of 15 to 21 nucleotides.

In some embodiments, the oligonucleotide, or a contiguous nucleotide sequence thereof, comprises or consists of 24 or less nucleotides, such as 22, such as 20 or less nucleotides, such as 14, 15, 16, 17, 18, 19, 20 or 21 nucleotides. It is to be understood that any range given herein includes the range endpoints. Accordingly, if a nucleic acid molecule is said to include from 15 to 20 nucleotides, both 15 and 20 nucleotide lengths are included.

In some embodiments, the contiguous nucleotide sequence comprises or consists of 12, 13, 14, 15, 16, 17, 18, 19, 20, 21 or 22 contiguous nucleotides in length.

The oligonucleotide(s) can modulate the expression of a target nucleic acid in a mammal or in a mammalian cell. In some embodiments, the nucleic acid molecules, such as for siRNAs, shRNAs and antisense oligonucleotides inhibit expression of a target nucleic acid(s).

In one embodiment of the invention, the oligonucleotide is selected from an RNAi agent, such as an siRNA or shRNA. In another embodiment, the oligonucleotide is a single stranded antisense oligonucleotide, such as a high affinity modified antisense oligonucleotide interacting with RNase H.

In some embodiments, the oligonucleotide of the invention may comprise one or more modified nucleosides or nucleotides, such as 2′ sugar modified nucleosides.

In some embodiments, the oligonucleotide comprises phosphorothioate internucleoside linkages.

A library of oligonucleotides is to be understood as a collection of different oligonucleotides. The purpose of the library of oligonucleotides can vary. In some embodiments, the library of oligonucleotides is composed of oligonucleotides with overlapping nucleobase sequence targeting one or more mammalian C4A and/or C4B target nucleic acids, designed for the purpose of identifying potent sequences, e.g., the most potent sequence, within the library of oligonucleotides. In some embodiments, the library of oligonucleotides is a library of oligonucleotide design variants (child nucleic acid molecules) of a parent or ancestral oligonucleotide, wherein the oligonucleotide design variants retain a core nucleobase sequence of the parent nucleic acid molecule, e.g., a conserved sequence of the parent.

Antisense Oligonucleotides

The term “antisense oligonucleotide” or “ASO” as used herein is defined as oligonucleotides capable of hybridizing to a target nucleic acid, in particular to a contiguous sequence on a target nucleic acid, e.g., to modulate expression of the corresponding target gene. Generally, nucleic acid molecules of the invention are antisense nucleic acids. The antisense oligonucleotides are not essentially double stranded and need not be siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), e.g., where the degree of intra or inter self-complementarity is less than 50% across of the full length of the oligonucleotide.

Advantageously, in some embodiments, the single stranded antisense oligonucleotide of the invention does not contain RNA nucleosides, since this will decrease nuclease resistance.

Advantageously, in some embodiments, the oligonucleotide of the invention comprises one or more modified nucleosides or nucleotides, such as 2′ sugar modified nucleosides. Furthermore, it is advantageous that, some, most, or all of the nucleosides, which are not modified, are DNA nucleosides, e.g., 50%, 75%, 95%, or 100% of the nucleosides which are not modified are DNA nucleosides.

RNAi Molecules

Herein, the term “RNA interference (RNAi) molecule” refers to short double-stranded oligonucleotide containing RNA nucleosides and which mediates targeted cleavage of an RNA transcript, e.g., via the RNA-induced silencing complex (RISC), where they interact with the catalytic RISC component argonaute. The RNAi molecule modulates, e.g., inhibits, the expression of the target nucleic acid in a cell, e.g. a cell within a subject, such as a mammalian subject. RNAi molecules includes single stranded RNAi molecules (Lima at al 2012 Cell 150: 883) and double stranded molecules, e.g., siRNAs or partially double-stranded molecules, as well as short hairpin RNAs (shRNAs). In some embodiments of the invention, the oligonucleotide of the invention or contiguous nucleotide sequence thereof is a RNAi agent, such as a siRNA.

siRNA

The term “small interfering ribonucleic acid” or “siRNA” refers to a small interfering ribonucleic acid RNAi molecule that generally interferes with the expression of an mRNA. The term refers to a class of double-stranded RNA molecules, also known in the art as short interfering RNA or silencing RNA. siRNAs typically comprise a sense strand (also referred to as a passenger strand) and an antisense strand (also referred to as the guide strand), wherein one or both strands are of 17 to 30 nucleotides in length, typically 19 to 25 nucleosides in length, wherein the antisense strand is complementary, such as at least 90%, e.g., 90-95% complementary, or such as fully complementary, to the target nucleic acid (suitably a mature mRNA sequence), and the sense strand is complementary to the antisense strand so that the sense strand and antisense strand form a duplex or duplex region. siRNA strands may form a blunt ended duplex, or advantageously the sense and/or antisense strand 3′ end may form a 3′ overhang of, e.g. 1, 2, or 3 nucleosides (e.g., to resemble the product produced by Dicer, which forms the RISC substrate in vivo. Effective extended forms of Dicer substrates have been described in U.S. Pat. Nos. 8,349,809 and 8,513,207, hereby incorporated by reference. In some embodiments, both the sense strand and antisense strand have a 2nt 3′ overhang. The duplex region may therefore be, for example 17 to 25 nucleotides in length, such as 21 to 23 nucleotide in length.

Once inside a cell the antisense strand can be incorporated into the RISC complex, which mediate target degradation or target inhibition of the target nucleic acid. siRNAs typically comprise modified nucleosides in addition to RNA nucleosides. In one embodiment, the siRNA molecule may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA. In particular, 2′fluoro, 2′-O-methyl or 2′-O-methoxyethyl may be incorporated into siRNAs.

In some embodiments, some, most, or all (e.g., 75-90%, 80-95%, 90-99%, or 100%) of the nucleotides of an siRNA sense (passenger) strand may be modified with 2′ sugar modified nucleosides such as LNA (see WO2004/083430 and WO2007/085485, for example). In some embodiments, the passenger stand of the siRNA may be discontinuous (see WO2007/107162 for example). In some embodiments, thermally destabilizing nucleotides at a seed region of the antisense strand of siRNAs are useful in reducing off-target activity of the siRNAs (see WO2018/098328 for example). In some embodiments, the siRNA comprises a 5′ phosphate group or a 5′-phosphate mimic at the 5′ end of the antisense strand. In some embodiments, the 5′ end of the antisense strand is a RNA nucleoside.

In one embodiment, the siRNA molecule further comprises at least one phosphorothioate or methylphosphonate internucleoside linkage. The phosphorothioaie or methylphosphonate internucleoside linkage may be at the 3′-terminus of one or both strands (e.g., the antisense strand and/or the sense strand); or the phosphorothioate or methylphosphonate internucleoside linkage may be at the 5′-terminus of one or both strands (e.g., the antisense strand and/or the sense strand); or the phosphorothioate or methylphosphonate internucleoside linkage may be at both the 5′- and 3′-termini of one or both strands (e.g., the antisense strand and/or the sense strand). In some embodiments, the remaining internucleoside linkages are phosphodiester linkages. In some embodiments, the siRNA molecule comprises one or more phosphorothioate internucleoside linkages. In siRNA molecules, phosphorothioate internucleoside linkages may reduce or inhibit nuclease cleavage in RICS. Accordingly, in some embodiments, not all internucleoside linkages in the antisense strand are modified, e.g., in some embodiments, 10-90%, 20-80%, 30-70%, or 40-60% of internucleoside linkages in the antisense strand are modified.

The siRNA molecule may further comprise a ligand. In some embodiments, the ligand is conjugated to the 3′ end of the sense strand.

For biological distribution, siRNAs may be conjugated to a targeting ligand, and/or be formulated into lipid nanoparticles. In a particular example, the nucleic acid molecule is conjugated to a moiety that targets a brain cell or other cell of the CNS. Thus, the nucleic acid molecule may be conjugated to a moiety that facilitates delivery across the blood brain barrier. For example, the nucleic acid molecule may be conjugated to an antibody or antibody fragment targeting the transferrin receptor.

Other aspects of the invention relate to pharmaceutical compositions, in particular, pharmaceutical compositions comprising dsRNA, such as siRNA molecules suitable for therapeutic use, and methods of inhibiting the expression of a target gene by administering the dsRNA molecules such as siRNAs of the invention, e.g., for the treatment of various disease conditions as disclosed herein.

shRNA

The term “short hairpin RNA” or “shRNA” refers to molecules that are generally between 40 and 70 nucleotides in length, such as between 45 and 65 nucleotides in length, such as 50 and 60 nucleotides in length and form a stem loop (hairpin) RNA structure which can interact with the endonuclease known as Dicer (believed to processes dsRNA into 19-23 base pair short interfering RNAs with characteristic two base 3′ overhangs which then can be incorporated into an RNA-induced silencing complex (RISC)). Upon binding to the appropriate target mRNA, one or more endonucleases within the RISC cleave the target to induce silencing. shRNA oligonucleotides may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA. In some embodiments, an shRNA molecule comprises one or more phosphorothioate internucleoside linkages. In RNAi molecules, phosphorothioate internucleoside linkages may reduce or inhibit nuclease cleavage in RICS. Accordingly, not all internucleoside linkages in the stem loop of the shRNA molecule are modified, e.g., in some embodiments, 10-90%, 20-80%, 30-70%, or 40-60% of internucleoside linkages in the antisense strand are modified. Phosphorothioate internucleoside linkages can advantageously be placed in the 3′ and/or 5′ end of the stem loop of the shRNA molecule, in particular, in the part of the molecule that is not complementary to the target nucleic acid. The region of the shRNA molecule that is complementary to the target nucleic acid may however also be modified, e.g., in the first 2 to 3 internucleoside linkages in the part that is predicted to become the 3′ and/or 5′ terminal following cleavage by Dicer.

Contiguous Nucleotide Sequence

The term “contiguous nucleotide sequence” refers to the region of the nucleic acid molecule, which is complementary to the target nucleic acid. The term is used interchangeably herein with the term “contiguous nucleobase sequence” and the term “oligonucleotide motif sequence”. In some embodiments, all the nucleotides of the oligonucleotide constitute the contiguous nucleotide sequence. In some embodiments, the contiguous nucleotide sequence is included in the guide strand of an siRNA molecule. In some embodiments, the contiguous nucleotide sequence is the part of an shRNA molecule, which is 95%, 98%, 99%, or 100% complementary to the target nucleic acid. In some embodiments, the oligonucleotide comprises the contiguous nucleotide sequence, such as a F-G-F′ gapmer region, and may optionally comprise further nucleotide(s), for example, a nucleotide linker region which may be used to attach a functional group (e.g. a conjugate group for targeting) to the contiguous nucleotide sequence. The nucleotide linker region may or may not be complementary to the target nucleic acid. In some embodiments, the nucleobase sequence of the antisense oligonucleotide is the contiguous nucleotide sequence. In some embodiments, the contiguous nucleotide sequence is 100% complementary to the target nucleic acid.

Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. Advantageously, in some embodiments, one or more of the modified nucleoside comprises a modified sugar moiety. The term “modified nucleoside” may also be used herein interchangeably with the term “nucleoside analogue” or “modified unit” or “modified monomer”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Internucleoside Linkage

The term “modified internucleoside linkage” is defined as generally understood by the skilled person, such as, as being a linkage other than phosphodiester (PO) linkages, that covalently couples two nucleosides together. The oligonucleotides of the invention may therefore comprise one or more modified internucleoside linkages, such as a one or more phosphorothioate internucleoside linkages, or one or more phosphorodithioate internucleoside linkages.

With the oligonucleotide of the invention, it can be advantageous to use phosphorothioate internucleoside linkages, e.g., for 10-90%, 20-80%, 30-70%, or 40-60% of internucleoside linkages.

Phosphorothioate internucleoside linkages are particularly useful due to nuclease resistance, beneficial pharmacokinetics, and ease of manufacture. In some embodiments, at least 50% of the internucleoside linkages in the oligonucleotide, or contiguous nucleotide sequence thereof, are phosphorothioate, such as at least 60%, e.g., 60-80%; such as at least 70%, e.g., 70-85%; such as at least 75%, e.g., 75-90%; such as at least 80%, e.g. 80-95%; or such as at least 90%, e.g., 90-99%, of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate.

In some advantageous embodiments, all the internucleoside linkages of the contiguous nucleotide sequence of the oligonucleotide are phosphorothioate, or all the internucleoside linkages of the oligonucleotide are phosphorothioate linkages.

In some embodiments, the antisense oligonucleotides may comprise other internucleoside linkages (other than phosphodiester and phosphorothioate), for example alkyl phosphonate/methyl phosphonate internucleoside linkages, which may be tolerated in an otherwise DNA phosphorothioate gap region (e.g., as in EP 2 742 135).

Nucleobase

The term “nucleobase” includes the purine (e.g. adenine and guanine) and pyrimidine (e.g. uracil, thymine and cytosine) moiety present in nucleosides and nucleotides, which form hydrogen bonds in nucleic acid hybridization. In the context of the present invention, the term nucleobase also encompasses modified nucleobases, which may differ from naturally occurring nucleobases, but are functional during nucleic acid hybridization. In this context, “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term “modified oligonucleotide” describes an oligonucleotide comprising one or more sugar-modified nucleosides and/or modified internucleoside linkages and/or modified nucleobases. The term “chimeric oligonucleotide” is a term that has been used in the literature to describe oligonucleotides comprising modified nucleosides and DNA nucleosides. The antisense oligonucleotide of the invention is advantageously a chimeric oligonucleotide.

Complementarity

The term “complementarity” or “complementary” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison, a nucleobase/nucleotide, which does not align (form a base pair), is termed a mismatch. Insertions and deletions are not allowed in the calculation of complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as referring to two nucleic acid strands (e.g. an oligonucleotide and a target nucleic acid) forming hydrogen bonds between base pairs on opposite strands, thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions, Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (Kd) of the reaction by ΔG°=−RT ln(Kd), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions, ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating a nucleic acid target by hybridization, oligonucleotides of the present invention hybridize to a target nucleic acid with estimated ΔG° values below −10 kcal/mol for oligonucleotides that are 10 to 30 nucleotides in length. In some embodiments, the degree or strength of hybridization is measured by the standard state Gibbs free energy ΔG°. The oligonucleotides may hybridize to a target nucleic acid with estimated ΔG° values below −10 kcal/mol, such as below −15 kcal/mol, such as below −20 kcal/mol and such as below −25 kcal/mol for oligonucleotides that are 8 to 30 nucleotides in length. In some embodiments, the oligonucleotides hybridize to a target nucleic acid with an estimated ΔG° value in the range of −10 to −60 kcal/mol, such as −12 to −40, such as from −15 to −30 kcal/mol or −16 to −27 kcal/mol such as −18 to −25 kcal/mol.

Target Nucleic Acid

According to the present invention, the target nucleic acid is a nucleic acid, which encodes a mammalian C4A or a mammalian C4B and may for example be a gene, a RNA, a mRNA, and pre-mRNA, a mature mRNA or a cDNA sequence. The target may therefore be referred to as C4A target nucleic acid or C4B target nucleic acid.

The therapeutic oligonucleotides of the invention may for example target exon regions of a mammalian C4A and/or C4B (in particular siRNA and shRNA, but also antisense oligonucleotides), or may for example target any intron region in the C4A and/or C4B pre-mRNA (in particular antisense oligonucleotides).

Table 1a and 1 b list predicted exon and intron regions of SEQ ID NO: 3 and 4, i.e. of the human C4A and C4B pre-mRNA sequence.

TABLE 1a
Exons and introns in the human C4A pre-mRNA.

| Exemplary exonic regions in the | | | Exemplary intronic regions in the | | |
|---|---|---|---|---|---|
| human C4A premRNA (SEQ ID | | | human C4A premRNA (SEQ ID | | |
| NO 3) | | | NO 3) | | |
| ID | start | end | ID | start | end |
| Ea1 | 1 | 149 | Ia1 | 150 | 281 |
| Ea2 | 282 | 480 | Ia2 | 481 | 694 |
| Ea3 | 695 | 896 | Ia3 | 897 | 1105 |
| Ea4 | 1106 | 1176 | Ia4 | 1177 | 1254 |
| Ea5 | 1255 | 1343 | Ia5 | 1344 | 1561 |
| Ea6 | 1562 | 1644 | Ia6 | 1645 | 1814 |
| Ea7 | 1815 | 1911 | Ia7 | 1912 | 2049 |
| Ea8 | 2050 | 2155 | Ia8 | 2156 | 2252 |
| Ea9 | 2253 | 2385 | Ia9 | 2386 | 9168 |
| Ea10 | 9169 | 9284 | Ia10 | 9285 | 9383 |
| Ea11 | 9384 | 9563 | Ia11 | 9564 | 9708 |
| Ea12 | 9709 | 9891 | Ia12 | 9892 | 10023 |
| Ea13 | 10024 | 10209 | Ia13 | 10210 | 10362 |
| Ea14 | 10363 | 10521 | Ia14 | 10522 | 10772 |
| Ea15 | 10773 | 10899 | Ia15 | 10900 | 11066 |
| Ea16 | 11067 | 11141 | Ia16 | 11142 | 11401 |
| Ea17 | 11402 | 11599 | Ia17 | 11600 | 11690 |
| Ea18 | 11691 | 11802 | Ia18 | 11803 | 11892 |
| Ea19 | 11893 | 11963 | Ia19 | 11964 | 12221 |
| Ea20 | 12222 | 12361 | Ia20 | 12362 | 12474 |
| Ea21 | 12475 | 12684 | Ia21 | 12685 | 12928 |
| Ea22 | 12929 | 12980 | Ia22 | 12981 | 13081 |
| Ea23 | 13082 | 13171 | Ia23 | 13172 | 13306 |
| Ea24 | 13307 | 13516 | Ia24 | 13517 | 13695 |
| Ea25 | 13696 | 13771 | Ia25 | 13772 | 13931 |
| Ea26 | 13932 | 14088 | Ia26 | 14089 | 14183 |
| Ea27 | 14184 | 14300 | Ia27 | 14301 | 14405 |
| Ea28 | 14406 | 14577 | Ia28 | 14578 | 14805 |
| Ea29 | 14806 | 15038 | Ia29 | 15039 | 15120 |
| Ea30 | 15121 | 15288 | Ia30 | 15289 | 15681 |
| Ea31 | 15682 | 15741 | Ia31 | 15742 | 16790 |
| Ea32 | 16791 | 16884 | Ia32 | 16885 | 16981 |
| Ea33 | 16982 | 17168 | Ia33 | 17169 | 17282 |
| Ea34 | 17283 | 17373 | Ia34 | 17374 | 17463 |
| Ea35 | 17464 | 17538 | Ia35 | 17539 | 19029 |
| Ea36 | 19030 | 19132 | Ia36 | 19133 | 19297 |
| Ea37 | 19298 | 19387 | Ia37 | 19388 | 19472 |
| Ea38 | 19473 | 19571 | Ia38 | 19572 | 19753 |
| Ea39 | 19754 | 19837 | Ia39 | 19838 | 20099 |
| Ea40 | 20100 | 20232 | Ia40 | 20233 | 20375 |
| Ea41 | 20376 | 20658 | | | |

TABLE 1b
Exons and introns in the human C4B pre-mRNA.

| Exemplary exonic regions in the | | | Exemplary intronic regions in the | | |
|---|---|---|---|---|---|
| human C4B premRNA (SEQ ID | | | human C4B premRNA (SEQ ID | | |
| NO 4) | | | NO 4) | | |
| ID | start | end | ID | start | end |
| Eb1 | 1 | 116 | Ib1 | 117 | 248 |
| Eb2 | 249 | 447 | Ib2 | 448 | 661 |
| Eb3 | 662 | 863 | Ib3 | 864 | 1072 |
| Eb4 | 1073 | 1143 | Ib4 | 1144 | 1221 |
| Eb5 | 1222 | 1310 | Ib5 | 1311 | 1528 |
| Eb6 | 1529 | 1611 | Ib6 | 1612 | 1781 |
| Eb7 | 1782 | 1878 | Ib7 | 1879 | 2016 |
| Eb8 | 2017 | 2122 | Ib8 | 2123 | 2219 |
| Eb9 | 2220 | 2352 | Ib9 | 2353 | 9135 |
| Eb10 | 9136 | 9251 | Ib10 | 9252 | 9350 |
| Eb11 | 9351 | 9530 | Ib11 | 9531 | 9675 |
| Eb12 | 9676 | 9858 | Ib12 | 9859 | 9990 |
| Eb13 | 9991 | 10176 | Ib13 | 10177 | 10329 |
| Eb14 | 10330 | 10488 | Ib14 | 10489 | 10739 |
| Eb15 | 10740 | 10866 | Ib15 | 10867 | 11033 |
| Eb16 | 11034 | 11108 | Ib16 | 11109 | 11368 |
| Eb17 | 11369 | 11566 | Ib17 | 11567 | 11657 |
| Eb18 | 11658 | 11769 | Ib18 | 11770 | 11859 |
| Eb19 | 11860 | 11930 | Ib19 | 11931 | 12188 |
| Eb20 | 12189 | 12328 | Ib20 | 12329 | 12441 |
| Eb21 | 12442 | 12651 | Ib21 | 12652 | 12895 |
| Eb22 | 12896 | 12947 | Ib22 | 12948 | 13048 |
| Eb23 | 13049 | 13138 | Ib23 | 13139 | 13273 |
| Eb24 | 13274 | 13483 | Ib24 | 13484 | 13662 |
| Eb25 | 13663 | 13738 | Ib25 | 13739 | 13898 |
| Eb26 | 13899 | 14055 | Ib26 | 14056 | 14150 |
| Eb27 | 14151 | 14267 | Ib27 | 14268 | 14372 |
| Eb28 | 14373 | 14544 | Ib28 | 14545 | 14771 |
| Eb29 | 14772 | 15004 | Ib29 | 15005 | 15086 |
| Eb30 | 15087 | 15254 | Ib30 | 15255 | 15647 |
| Eb31 | 15648 | 15707 | Ib31 | 15708 | 16756 |
| Eb32 | 16757 | 16850 | Ib32 | 16851 | 16947 |
| Eb33 | 16948 | 17134 | Ib33 | 17135 | 17248 |
| Eb34 | 17249 | 17339 | Ib34 | 17340 | 17429 |
| Eb35 | 17430 | 17504 | Ib35 | 17505 | 18995 |
| Eb36 | 18996 | 19098 | Ib36 | 19099 | 19263 |
| Eb37 | 19264 | 19353 | Ib37 | 19354 | 19438 |
| Eb38 | 19439 | 19537 | Ib38 | 19538 | 19719 |
| Eb39 | 19720 | 19803 | Ib39 | 19804 | 20065 |
| Eb40 | 20066 | 20198 | Ib40 | 20199 | 20341 |
| Eb41 | 20342 | 20624 | | | |

In some embodiments, the target nucleic acid encodes a C4A protein, in particular a mammalian C4A protein, such as a human C4A protein. In some embodiments, the target nucleic acid encodes a C4B protein, in particular a mammalian C4B protein, such as a human C4B protein. See for example Table 2 and Table 3, which provides an overview on the genomic sequences of human, cyno monkey and mouse C4 (Table 2) and on pre-mRNA sequences for human, monkey and mouse C4 and for the mature mRNAs for human C4 (Table 3).

In some embodiments, the target nucleic acid is selected from the group consisting of SEQ ID NO: 1, 2, 3, 4, 5, 6, and 7, or naturally occurring variants thereof (e.g. sequences encoding a mammalian C4A and/or C4B).

TABLE 2
Genome and assembly information for C4A and C4B across species.

| | | | Genomic | | | NCBI reference |
|---|---|---|---|---|---|---|
| | | | coordinates | | | sequence* accession |
| Species | Chr | Strand | Start | End | Assembly | number for mRNA |
| Mouse | 17 | Rev | 34728380 | 34743882 | GRCm38.p6 | NM_009780.2 |
| Mouse | 17 | Rev | 34809092 | 34823464 | GRCm38.p6 | NM_011413.2 |
| Human | 6 | Fwd | 31982057 | 32002681 | GRCh38.p12 | NM_007293.3 and |
| | | | | | | NM_001252204.1 |
| Human | 6 | Fwd | 32014795 | 32035418 | GRCh38.p12 | NM_001002029.3 |
| Cyno | 4 | Rev | 138859330 | 138873551 | Macaca_fascicularis_5 | |

Fwd = forward strand.
Rev = reverse strand.
The genome coordinates provide the pre-mRNA sequence(genomic sequence).

If employing the nucleic acid molecule of the invention in research or diagnostics, the target nucleic acid may be a cDNA or a synthetic nucleic acid derived from DNA or RNA.

For in vivo or in vitro application, the therapeutic nucleic acid molecule of the invention is typically capable of inhibiting the expression of the C4A and/or C4B target nucleic acid in a cell, which is expressing the C4A and C4B target nucleic acid. The contiguous sequence of nucleobases of the nucleic acid molecule of the invention is typically complementary to a conserved region of the C4A and/or C4B target nucleic acid, as measured across the length of the nucleic acid molecule, optionally with the exception of one or two mismatches. In some embodiments, the target nucleic acid is a messenger RNA, such as a pre-mRNA which encodes mammalian C4A protein, such as mouse C4a, e.g. the mouse C4a pre-mRNA sequence, such as that disclosed as SEQ ID NO: 2, the human C4A pre-mRNA sequence, such as that disclosed as SEQ ID NO: 3, or the cyno monkey C4 pre-mRNA sequence, such as that disclosed as SEQ ID NO: 5, or a mature C4A mRNA, such as that of a human mature mRNA disclosed as SEQ ID NO: 6. In some embodiments, the target nucleic acid is a messenger RNA, such as a pre-mRNA which encodes mammalian C4B protein, such as mouse C4b, e.g. the mouse C4b pre-mRNA sequence, such as that disclosed as SEQ ID NO: 1, the human C4B pre-mRNA sequence, such as that disclosed as SEQ ID NO: 4, or the cyno monkey C4 pre-mRNA sequence, such as that disclosed as SEQ ID NO: 5, or a mature C4B mRNA, such as that of a human mature mRNA disclosed as SEQ ID NO:7. SEQ ID NOs: 1, 2, 3, 4, 5, 6, and 7, are DNA sequences—it will be understood that target RNA sequences have uracil (U) bases in place of the thymidine bases (T).

It is known that different, i.e. shorter, annotated mRNA isoforms of the above sequences exist. The isoforms are well-known in the art and can be derived from the known sequence databases.

Further information on exemplary target nucleic acids is provided in Table 3.

TABLE 3
Overview on target nucleic acids.

| Target Nucleic Acid, Species, Reference | Sequence ID |
|---|---|
| C4b Mus musculus pre-mRNA | SEQ ID NO: 1 |
| C4a Mus musculus pre-mRNA | SEQ ID NO: 2 |
| C4A Homo sapiens pre-mRNA | SEQ ID NO: 3 |
| C4B Homo sapiens pre-mRNA | SEQ ID NO: 4 |
| C4 Macaca fascicularis pre-mRNA | SEQ ID NO: 5 |
| C4A Homo sapiens mature mRNA | SEQ ID NO: 6 |
| C4B Homo sapiens mature mRNA | SEQ ID NO: 7 |

In some embodiments, the target nucleic acid is SEQ ID NO: 1.

In some embodiments, the target nucleic acid is SEQ ID NO: 2.

In some embodiments, the target nucleic acid is SEQ ID NO: 3.

In some embodiments, the target nucleic acid is SEQ ID NO: 4.

In some embodiments, the target nucleic acid is SEQ ID NO: 5.

In some embodiments, the target nucleic acid is SEQ ID NO: 6.

In some embodiments, the target nucleic acid is SEQ ID NO: 7.

Target

The term “target” as used herein refers to the complement component 4 (C4), which can in the context of this disclosure be C4A and/or C4B. Further, the term “target” can refer to the C4A target nucleic acid and/or C4B target nucleic acid, as well as the C4A protein and/or C4B protein. For example, part of the antisense oligonucleotides described herein target both C4A target nucleic acid and C4B target nucleic acid, i.e. such antisense oligonucleotides target both C4A target nucleic acid and C4B target nucleic acid by binding C4A/C4B homologous regions (pan-C4 antisense oligonucleotides). As known in the art, the terms “C4A” and “C4B” (uppercase A/B) relate to the human target. The terms “C4a” and “C4b” (lower case a/b) relate to the mouse target.

Target Sequence

The term “target sequence” as used herein refers to a sequence of nucleotides present in the target nucleic acid, which comprises the nucleobase sequence, which is complementary to the oligonucleotide or nucleic acid molecule of the invention. In some embodiments, the target sequence comprises or consists of a region on the target nucleic acid with a nucleobase sequence that is complementary to the contiguous nucleotide sequence of the oligonucleotide of the invention. This region of the target nucleic acid may interchangeably be referred to as the target nucleotide sequence, target sequence or target region. In some embodiments, the target sequence is longer than the complementary sequence of a nucleic acid molecule of the invention, and may, for example represent a preferred region of the target nucleic acid, which may be targeted by several nucleic acid molecules of the invention. It is well known in the art that C4A and C4B genes display high level of variability between individuals. The term “target sequence” encompasses all publicly annotated variants of C4A and C4B.

In some embodiments, the target sequence is a sequence selected from the group consisting of a human C4A mRNA exon, such as a human C4A mRNA exon selected from the group consisting of Ea1-Ea41 (see for example Table 1a above). In some embodiments, the target sequence is a sequence selected from the group consisting of a human C4B mRNA exon, such as a human C4B mRNA exon selected from the group consisting of Eb1-Eb41 (see for example Table 1 b above).

Accordingly, the invention provides for an oligonucleotide, wherein said oligonucleotide comprises a contiguous sequence which is at least 90% complementary, such as 90-95% or fully complementary, to an exon region of SEQ ID NO: 3 and 4, selected from the group consisting of Ea1-Ea41 and Eb1-Eb41 (see Table 1a and 1 b).

In some embodiments, the target sequence is a sequence selected from the group consisting of a human C4A mRNA intron, such as a human C4A mRNA intron selected from the group consisting of Ia1-Ia40 (see for example Table 1a above). In some embodiments, the target sequence is a sequence selected from the group consisting of a human C4B mRNA exon, such as a human C4B mRNA intron selected from the group consisting of Ib1-Ib40 (see for example Table 1 b above).

Accordingly, the invention provides an oligonucleotide, wherein said oligonucleotide comprises a contiguous sequence which is at least 90% complementary, such as 90-95% or fully complementary, to an intron region of SEQ ID NO: 3 and 4, selected from the group consisting of Ia1-Ia40 and Ib1-Ib40 (see Table(s) la and 1b).

In some embodiments, the target sequence is selected from the group consisting of SEQ ID NO: 6, and 7. In some embodiments, the contiguous nucleotide sequence as referred to herein is at least 90% (e.g., 90-95%) complementary, such as at least 95% (e.g., 95-98) complementary to a target sequence selected from the group consisting of SEQ ID NO: 6, and 7. In some embodiments, the contiguous nucleotide sequence is fully complementary to a target sequence selected from the group consisting of SEQ ID NO: 6, and 7.

The oligonucleotide of the invention comprises a contiguous nucleotide sequence, which is complementary to or hybridizes to a region on the target nucleic acid, such as a target sequence described herein.

The target nucleic acid sequence to which the oligonucleotide is complementary or hybridizes to generally comprises a stretch of contiguous nucleobases of at least 10 nucleotides. The contiguous nucleotide sequence is between 12 to 70 nucleotides, such as 12 to 50, such as 13 to 30, such as 14 to 25, such as 15 to 21 contiguous nucleotides.

In some embodiments, the oligonucleotide of the present invention targets a region shown in Table 4a and 4b.

TABLE 4a
Exemplary target regions on SEQ ID NO: 3

| | start | end |
|---|---|---|
| Target | SEQ ID | SEQ ID |
| region | NO: 3 | NO: 3 |
| 1A | 22 | 65 |
| 2A | 74 | 92 |
| 3A | 94 | 131 |
| 4A | 133 | 196 |
| 5A | 200 | 230 |
| 6A | 248 | 390 |
| 7A | 405 | 452 |
| 8A | 454 | 485 |
| 9A | 496 | 516 |
| 10A | 567 | 591 |
| 11A | 612 | 640 |
| 12A | 648 | 670 |
| 13A | 678 | 702 |
| 14A | 712 | 768 |
| 15A | 770 | 800 |
| 16A | 819 | 852 |
| 17A | 854 | 922 |
| 18A | 945 | 980 |
| 19A | 982 | 997 |
| 20A | 1031 | 1046 |
| 21A | 1056 | 1072 |
| 22A | 1074 | 1091 |
| 23A | 1099 | 1133 |
| 24A | 1135 | 1168 |
| 25A | 1170 | 1236 |
| 26A | 1238 | 1286 |
| 27A | 1295 | 1349 |
| 28A | 1351 | 1368 |
| 29A | 1370 | 1384 |
| 30A | 1386 | 1431 |
| 31A | 1444 | 1485 |
| 32A | 1487 | 1501 |
| 33A | 1504 | 1524 |
| 34A | 1541 | 1612 |
| 35A | 1614 | 1673 |
| 36A | 1675 | 1701 |
| 37A | 1719 | 1737 |
| 38A | 1739 | 1756 |
| 39A | 1780 | 1890 |
| 40A | 1892 | 1938 |
| 41A | 1950 | 1966 |
| 42A | 1968 | 1989 |
| 43A | 1998 | 2040 |
| 44A | 2042 | 2100 |
| 45A | 2102 | 2128 |
| 46A | 2130 | 2173 |
| 47A | 2175 | 2204 |
| 48A | 2226 | 2302 |
| 49A | 2312 | 2329 |
| 50A | 2342 | 2418 |
| 51A | 2422 | 2452 |
| 52A | 2487 | 2522 |
| 53A | 2538 | 2562 |
| 54A | 2606 | 2638 |
| 55A | 2649 | 2668 |
| 56A | 3229 | 3243 |
| 57A | 3231 | 3245 |
| 58A | 9028 | 9045 |
| 59A | 9047 | 9070 |
| 60A | 9072 | 9099 |
| 61A | 9108 | 9130 |
| 62A | 9142 | 9161 |
| 63A | 9163 | 9204 |
| 64A | 9206 | 9317 |
| 65A | 9320 | 9348 |
| 66A | 9350 | 9445 |
| 67A | 9447 | 9477 |
| 68A | 9501 | 9526 |
| 69A | 9528 | 9562 |
| 70A | 9564 | 9618 |
| 71A | 9595 | 9611 |
| 72A | 9665 | 9687 |
| 73A | 9701 | 9725 |
| 74A | 9736 | 9911 |
| 75A | 9921 | 9945 |
| 76A | 9956 | 9971 |
| 77A | 9973 | 10010 |
| 78A | 10019 | 10074 |
| 79A | 10090 | 10153 |
| 80A | 10155 | 10214 |
| 81A | 10226 | 10241 |
| 82A | 10250 | 10295 |
| 83A | 10309 | 10327 |
| 84A | 10352 | 10389 |
| 85A | 10393 | 10430 |
| 86A | 10440 | 10540 |
| 87A | 10562 | 10577 |
| 88A | 10589 | 10613 |
| 89A | 10635 | 10662 |
| 90A | 10665 | 10685 |
| 91A | 10693 | 10712 |
| 92A | 10741 | 10763 |
| 93A | 10764 | 10782 |
| 94A | 10784 | 10798 |
| 95A | 10800 | 10876 |
| 96A | 10890 | 10931 |
| 97A | 10933 | 10963 |
| 98A | 10987 | 11008 |
| 99A | 11001 | 11017 |
| 100A | 11012 | 11054 |
| 101A | 11056 | 11070 |
| 102A | 11073 | 11099 |
| 103A | 11114 | 11149 |
| 104A | 11166 | 11181 |
| 105A | 11200 | 11219 |
| 106A | 11238 | 11277 |
| 107A | 11284 | 11303 |
| 108A | 11305 | 11337 |
| 109A | 11339 | 11375 |
| 110A | 11377 | 11420 |
| 111A | 11422 | 11467 |
| 112A | 11469 | 11495 |
| 113A | 11497 | 11566 |
| 114A | 11586 | 11634 |
| 115A | 11637 | 11655 |
| 116A | 11657 | 11671 |
| 117A | 11673 | 11812 |
| 118A | 11870 | 11925 |
| 119A | 11939 | 11967 |
| 120A | 11972 | 11996 |
| 121A | 12017 | 12054 |
| 122A | 12086 | 12116 |
| 123A | 12118 | 12142 |
| 124A | 12155 | 12195 |
| 125A | 12213 | 12235 |
| 126A | 12251 | 12347 |
| 127A | 12352 | 12367 |
| 128A | 12375 | 12392 |
| 129A | 12394 | 12409 |
| 130A | 12411 | 12428 |
| 131A | 12454 | 12572 |
| 132A | 12574 | 12593 |
| 133A | 12595 | 12611 |
| 134A | 12613 | 12641 |
| 135A | 12643 | 12659 |
| 136A | 12667 | 12706 |
| 137A | 12719 | 12748 |
| 138A | 12750 | 12784 |
| 139A | 12807 | 12855 |
| 140A | 12875 | 12902 |
| 141A | 12904 | 12947 |
| 142A | 12949 | 12976 |
| 143A | 12989 | 13006 |
| 144A | 13008 | 13106 |
| 145A | 13108 | 13160 |
| 146A | 13162 | 13188 |
| 147A | 13190 | 13210 |
| 148A | 13227 | 13267 |
| 149A | 13284 | 13328 |
| 150A | 13330 | 13349 |
| 151A | 13351 | 13375 |
| 152A | 13386 | 13467 |
| 153A | 13477 | 13496 |
| 154A | 13498 | 13526 |
| 155A | 13530 | 13546 |
| 156A | 13548 | 13566 |
| 157A | 13568 | 13594 |
| 158A | 13621 | 13642 |
| 159A | 13644 | 13692 |
| 160A | 13694 | 13758 |
| 161A | 13799 | 13813 |
| 162A | 13857 | 13874 |
| 163A | 13892 | 13918 |
| 164A | 13924 | 13947 |
| 165A | 13949 | 14009 |
| 166A | 14017 | 14042 |
| 167A | 14056 | 14070 |
| 168A | 14082 | 14105 |
| 169A | 14145 | 14168 |
| 170A | 14201 | 14269 |
| 171A | 14285 | 14305 |
| 172A | 14308 | 14326 |
| 173A | 14394 | 14449 |
| 174A | 14457 | 14515 |
| 175A | 14532 | 14584 |
| 176A | 14608 | 14642 |
| 177A | 14644 | 14676 |
| 178A | 14749 | 14771 |
| 179A | 14803 | 14831 |
| 180A | 14833 | 14860 |
| 181A | 14881 | 14965 |
| 182A | 14967 | 14981 |
| 183A | 14988 | 15048 |
| 184A | 15064 | 15079 |
| 185A | 15109 | 15176 |
| 186A | 15190 | 15218 |
| 187A | 15244 | 15301 |
| 188A | 15323 | 15355 |
| 189A | 15381 | 15414 |
| 190A | 15418 | 15437 |
| 191A | 15439 | 15478 |
| 192A | 15514 | 15540 |
| 193A | 15554 | 15608 |
| 194A | 15610 | 15630 |
| 195A | 15643 | 15668 |
| 196A | 15679 | 15742 |
| 197A | 15794 | 15817 |
| 198A | 15819 | 15855 |
| 199A | 15857 | 15897 |
| 200A | 15915 | 15942 |
| 201A | 15966 | 15995 |
| 202A | 16003 | 16024 |
| 203A | 16026 | 16043 |
| 204A | 16045 | 16064 |
| 205A | 16068 | 16086 |
| 206A | 16126 | 16178 |
| 207A | 16180 | 16212 |
| 208A | 16244 | 16268 |
| 209A | 16280 | 16327 |
| 210A | 16329 | 16363 |
| 211A | 16385 | 16405 |
| 212A | 16456 | 16486 |
| 213A | 16497 | 16584 |
| 214A | 16589 | 16603 |
| 215A | 16674 | 16699 |
| 216A | 16724 | 16752 |
| 217A | 16762 | 16800 |
| 218A | 16802 | 16843 |
| 219A | 16845 | 16908 |
| 220A | 16941 | 17022 |
| 221A | 17049 | 17185 |
| 222A | 17187 | 17265 |
| 223A | 17274 | 17325 |
| 224A | 17327 | 17366 |
| 225A | 17368 | 17383 |
| 226A | 17387 | 17405 |
| 227A | 17415 | 17445 |
| 228A | 17448 | 17552 |
| 229A | 17558 | 17579 |
| 230A | 17595 | 17631 |
| 231A | 17690 | 17724 |
| 232A | 17789 | 17837 |
| 233A | 17841 | 17861 |
| 234A | 17874 | 17896 |
| 235A | 17901 | 17918 |
| 236A | 17930 | 17953 |
| 237A | 17960 | 17977 |
| 238A | 17970 | 17984 |
| 239A | 17987 | 18008 |
| 240A | 18038 | 18053 |
| 241A | 18055 | 18074 |
| 242A | 18076 | 18095 |
| 243A | 18121 | 18170 |
| 244A | 18172 | 18190 |
| 245A | 18207 | 18269 |
| 246A | 18358 | 18376 |
| 247A | 18389 | 18423 |
| 248A | 18418 | 18455 |
| 249A | 18462 | 18495 |
| 250A | 18515 | 18552 |
| 251A | 18582 | 18596 |
| 252A | 18606 | 18621 |
| 253A | 18638 | 18652 |
| 254A | 18654 | 18668 |
| 255A | 18680 | 18713 |
| 256A | 18715 | 18729 |
| 257A | 18731 | 18761 |
| 258A | 18776 | 18807 |
| 259A | 18824 | 18875 |
| 260A | 18877 | 18895 |
| 261A | 18901 | 18933 |
| 262A | 18972 | 19022 |
| 263A | 19024 | 19076 |
| 264A | 19081 | 19107 |
| 265A | 19109 | 19130 |
| 266A | 19171 | 19188 |
| 267A | 19230 | 19259 |
| 268A | 19288 | 19344 |
| 269A | 19346 | 19423 |
| 270A | 19438 | 19458 |
| 271A | 19466 | 19616 |
| 272A | 19630 | 19648 |
| 273A | 19650 | 19667 |
| 274A | 19677 | 19698 |
| 275A | 19713 | 19728 |
| 276A | 19742 | 19763 |
| 277A | 19765 | 19790 |
| 278A | 19792 | 19846 |
| 279A | 19853 | 19870 |
| 280A | 19872 | 19893 |
| 281A | 19900 | 19934 |
| 282A | 19966 | 19990 |
| 283A | 19992 | 20019 |
| 284A | 20048 | 20065 |
| 285A | 20067 | 20081 |
| 286A | 20083 | 20116 |
| 287A | 20132 | 20151 |
| 288A | 20156 | 20180 |
| 289A | 20182 | 20239 |
| 290A | 20246 | 20260 |
| 291A | 20264 | 20294 |
| 292A | 20296 | 20320 |
| 293A | 20330 | 20420 |
| 294A | 20422 | 20462 |
| 295A | 20474 | 20530 |
| 296A | 20554 | 20585 |
| 297A | 20597 | 20629 |

TABLE 4b
Exemplary target regions on SEQ ID NO: 4

| | start | end |
|---|---|---|
| Target | SEQ ID | SEQ ID |
| region | NO: 4 | NO: 4 |
| 1B | 1 | 32 |
| 2B | 41 | 59 |
| 3B | 61 | 98 |
| 4B | 100 | 163 |
| 5B | 167 | 197 |
| 6B | 215 | 357 |
| 7B | 372 | 419 |
| 8B | 421 | 452 |
| 9B | 463 | 483 |
| 10B | 534 | 558 |
| 11B | 579 | 607 |
| 12B | 615 | 637 |
| 13B | 645 | 669 |
| 14B | 679 | 735 |
| 15B | 737 | 767 |
| 16B | 786 | 819 |
| 17B | 821 | 889 |
| 18B | 912 | 947 |
| 19B | 949 | 964 |
| 20B | 998 | 1013 |
| 21B | 1023 | 1039 |
| 22B | 1041 | 1058 |
| 23B | 1066 | 1100 |
| 24B | 1102 | 1135 |
| 25B | 1137 | 1203 |
| 26B | 1205 | 1253 |
| 27B | 1262 | 1316 |
| 28B | 1318 | 1335 |
| 29B | 1337 | 1351 |
| 30B | 1353 | 1398 |
| 31B | 1411 | 1452 |
| 32B | 1454 | 1468 |
| 33B | 1471 | 1491 |
| 34B | 1508 | 1579 |
| 35B | 1581 | 1640 |
| 36B | 1642 | 1668 |
| 37B | 1686 | 1704 |
| 38B | 1706 | 1723 |
| 39B | 1747 | 1857 |
| 40B | 1859 | 1905 |
| 41B | 1917 | 1933 |
| 42B | 1935 | 1956 |
| 43B | 1965 | 2007 |
| 44B | 2009 | 2067 |
| 45B | 2069 | 2095 |
| 46B | 2097 | 2140 |
| 47B | 2142 | 2171 |
| 48B | 2193 | 2269 |
| 49B | 2279 | 2296 |
| 50B | 2309 | 2385 |
| 51B | 2389 | 2419 |
| 52B | 2454 | 2489 |
| 53B | 2505 | 2529 |
| 54B | 2573 | 2605 |
| 55B | 2616 | 2635 |
| 56B | 3196 | 3210 |
| 57B | 3198 | 3212 |
| 58B | 8995 | 9012 |
| 59B | 9014 | 9037 |
| 60B | 9039 | 9066 |
| 61B | 9075 | 9097 |
| 62B | 9109 | 9128 |
| 63B | 9130 | 9171 |
| 64B | 9173 | 9284 |
| 65B | 9287 | 9315 |
| 66B | 9317 | 9412 |
| 67B | 9414 | 9444 |
| 68B | 9468 | 9493 |
| 69B | 9495 | 9529 |
| 70B | 9531 | 9585 |
| 71B | 9562 | 9578 |
| 72B | 9632 | 9654 |
| 73B | 9668 | 9692 |
| 74B | 9703 | 9878 |
| 75B | 9888 | 9912 |
| 76B | 9923 | 9938 |
| 77B | 9940 | 9977 |
| 78B | 9986 | 10041 |
| 79B | 10057 | 10120 |
| 80B | 10122 | 10181 |
| 81B | 10193 | 10208 |
| 82B | 10217 | 10262 |
| 83B | 10276 | 10294 |
| 84B | 10319 | 10356 |
| 85B | 10360 | 10397 |
| 86B | 10407 | 10507 |
| 87B | 10529 | 10544 |
| 88B | 10556 | 10580 |
| 89B | 10602 | 10629 |
| 90B | 10632 | 10652 |
| 91B | 10660 | 10679 |
| 92B | 10708 | 10730 |
| 93B | 10731 | 10749 |
| 94B | 10751 | 10765 |
| 95B | 10767 | 10843 |
| 96B | 10857 | 10898 |
| 97B | 10900 | 10930 |
| 98B | 10954 | 10975 |
| 99B | 10968 | 10984 |
| 100B | 10979 | 11021 |
| 101B | 11023 | 11037 |
| 102B | 11040 | 11066 |
| 103B | 11081 | 11116 |
| 104B | 11133 | 11148 |
| 105B | 11167 | 11186 |
| 106B | 11205 | 11244 |
| 107B | 11251 | 11270 |
| 108B | 11272 | 11304 |
| 109B | 11306 | 11342 |
| 110B | 11344 | 11387 |
| 111B | 11389 | 11434 |
| 112B | 11436 | 11462 |
| 113B | 11464 | 11533 |
| 114B | 11553 | 11601 |
| 115B | 11604 | 11622 |
| 116B | 11624 | 11638 |
| 117B | 11640 | 11779 |
| 118B | 11837 | 11892 |
| 119B | 11906 | 11934 |
| 120B | 11939 | 11963 |
| 121B | 11984 | 12021 |
| 122B | 12053 | 12083 |
| 123B | 12085 | 12109 |
| 124B | 12122 | 12162 |
| 125B | 12180 | 12202 |
| 126B | 12218 | 12314 |
| 127B | 12319 | 12334 |
| 128B | 12341 | 12359 |
| 129B | 12361 | 12376 |
| 130B | 12378 | 12395 |
| 131B | 12421 | 12539 |
| 132B | 12541 | 12560 |
| 133B | 12580 | 12608 |
| 134B | 12610 | 12626 |
| 135B | 12634 | 12673 |
| 136B | 12686 | 12715 |
| 137B | 12717 | 12751 |
| 138B | 12774 | 12822 |
| 139B | 12842 | 12869 |
| 140B | 12871 | 12914 |
| 141B | 12916 | 12943 |
| 142B | 12956 | 12973 |
| 143B | 12975 | 13073 |
| 144B | 13075 | 13127 |
| 145B | 13129 | 13155 |
| 146B | 13157 | 13177 |
| 147B | 13194 | 13234 |
| 148B | 13251 | 13295 |
| 149B | 13297 | 13316 |
| 150B | 13318 | 13342 |
| 151B | 13353 | 13434 |
| 152B | 13444 | 13463 |
| 153B | 13465 | 13493 |
| 154B | 13497 | 13513 |
| 155B | 13515 | 13533 |
| 156B | 13535 | 13561 |
| 157B | 13588 | 13609 |
| 158B | 13611 | 13659 |
| 159B | 13661 | 13735 |
| 160B | 13766 | 13780 |
| 161B | 13824 | 13841 |
| 162B | 13859 | 13885 |
| 163B | 13891 | 13914 |
| 164B | 13916 | 13976 |
| 165B | 13984 | 14009 |
| 166B | 14049 | 14072 |
| 167B | 14112 | 14135 |
| 168B | 14168 | 14236 |
| 169B | 14252 | 14272 |
| 170B | 14275 | 14293 |
| 171B | 14361 | 14394 |
| 172B | 14396 | 14416 |
| 173B | 14424 | 14482 |
| 174B | 14489 | 14551 |
| 175B | 14574 | 14608 |
| 176B | 14610 | 14642 |
| 177B | 14715 | 14737 |
| 178B | 14769 | 14797 |
| 179B | 14799 | 14826 |
| 180B | 14847 | 14931 |
| 181B | 14933 | 14947 |
| 182B | 14954 | 15014 |
| 183B | 15030 | 15045 |
| 184B | 15075 | 15142 |
| 185B | 15156 | 15184 |
| 186B | 15210 | 15267 |
| 187B | 15289 | 15321 |
| 188B | 15347 | 15380 |
| 189B | 15384 | 15403 |
| 190B | 15409 | 15444 |
| 191B | 15480 | 15506 |
| 192B | 15520 | 15548 |
| 193B | 15550 | 15574 |
| 194B | 15576 | 15596 |
| 195B | 15609 | 15634 |
| 196B | 15645 | 15708 |
| 197B | 15760 | 15783 |
| 198B | 15785 | 15821 |
| 199B | 15823 | 15863 |
| 200B | 15881 | 15908 |
| 201B | 15932 | 15961 |
| 202B | 15969 | 15990 |
| 203B | 15992 | 16009 |
| 204B | 16011 | 16030 |
| 205B | 16034 | 16052 |
| 206B | 16092 | 16144 |
| 207B | 16146 | 16178 |
| 208B | 16210 | 16234 |
| 209B | 16246 | 16293 |
| 210B | 16295 | 16329 |
| 211B | 16351 | 16371 |
| 212B | 16422 | 16452 |
| 213B | 16463 | 16550 |
| 214B | 16555 | 16569 |
| 215B | 16640 | 16665 |
| 216B | 16690 | 16718 |
| 217B | 16728 | 16766 |
| 218B | 16768 | 16809 |
| 219B | 16811 | 16874 |
| 220B | 16907 | 16988 |
| 221B | 17015 | 17151 |
| 222B | 17153 | 17231 |
| 223B | 17240 | 17291 |
| 224B | 17293 | 17332 |
| 225B | 17334 | 17349 |
| 226B | 17353 | 17371 |
| 227B | 17381 | 17411 |
| 228B | 17414 | 17518 |
| 229B | 17524 | 17545 |
| 230B | 17561 | 17597 |
| 231B | 17656 | 17690 |
| 232B | 17755 | 17803 |
| 233B | 17807 | 17827 |
| 234B | 17840 | 17862 |
| 235B | 17867 | 17884 |
| 236B | 17896 | 17919 |
| 237B | 17926 | 17943 |
| 238B | 17936 | 17950 |
| 239B | 17953 | 17974 |
| 240B | 18004 | 18019 |
| 241B | 18021 | 18040 |
| 242B | 18042 | 18061 |
| 243B | 18087 | 18136 |
| 244B | 18138 | 18156 |
| 245B | 18173 | 18235 |
| 246B | 18324 | 18342 |
| 247B | 18355 | 18389 |
| 248B | 18384 | 18421 |
| 249B | 18428 | 18461 |
| 250B | 18481 | 18518 |
| 251B | 18548 | 18562 |
| 252B | 18572 | 18587 |
| 253B | 18604 | 18618 |
| 254B | 18620 | 18634 |
| 255B | 18646 | 18679 |
| 256B | 18681 | 18695 |
| 257B | 18697 | 18727 |
| 258B | 18742 | 18773 |
| 259B | 18790 | 18841 |
| 260B | 18843 | 18861 |
| 261B | 18867 | 18899 |
| 262B | 18938 | 18988 |
| 263B | 18990 | 19042 |
| 264B | 19047 | 19073 |
| 265B | 19075 | 19096 |
| 266B | 19137 | 19154 |
| 267B | 19196 | 19225 |
| 268B | 19254 | 19310 |
| 269B | 19312 | 19389 |
| 270B | 19404 | 19424 |
| 271B | 19432 | 19582 |
| 272B | 19596 | 19614 |
| 273B | 19616 | 19633 |
| 274B | 19643 | 19664 |
| 275B | 19679 | 19694 |
| 276B | 19708 | 19729 |
| 277B | 19731 | 19756 |
| 278B | 19758 | 19812 |
| 279B | 19819 | 19836 |
| 280B | 19838 | 19859 |
| 281B | 19866 | 19900 |
| 282B | 19932 | 19956 |
| 283B | 19958 | 19985 |
| 284B | 20014 | 20031 |
| 285B | 20033 | 20047 |
| 286B | 20049 | 20082 |
| 287B | 20098 | 20117 |
| 288B | 20122 | 20146 |
| 289B | 20148 | 20205 |
| 290B | 20212 | 20226 |
| 291B | 20230 | 20260 |
| 292B | 20262 | 20286 |
| 293B | 20296 | 20386 |
| 294B | 20388 | 20428 |
| 295B | 20440 | 20496 |
| 296B | 20520 | 20551 |
| 297B | 20563 | 20595 |

Target Cell

The term “target cell” as used herein refers to a cell expressing the target nucleic acid. For the therapeutic use of the present invention, it is advantageous if the target cell is a brain cell. In some embodiments, the brain cell is selected from the group consisting of a neuron, an astrocyte, an oligodendrocyte, and a microglia cell. In some embodiments, the target cell may be in vivo or in vitro. In some embodiments, the target cell is a mammalian cell such as a rodent cell, such as a mouse cell or a rat cell, or a woodchuck cell, or a primate cell such as a monkey cell (e.g. a cynomolgus monkey cell) or a human cell.

In some embodiments, the target cell expresses C4A mRNA, such as the C4A pre-mRNA or C4A mature mRNA. In some embodiments, the target cell expresses C4B mRNA, such as the C4B pre-mRNA or C4B mature mRNA. The poly A tail of the C4A mRNA or the C4B mRNA is typically disregarded for antisense oligonucleotide targeting.

Naturally Occurring Variant

The term “naturally occurring variant” refers to variants of the C4A and/or C4B gene or transcripts which originate from the same genetic loci as the target nucleic acid, but may differ, for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficiently complementary sequence of the oligonucleotide, the oligonucleotide of the invention may therefore target the target nucleic acid and naturally occurring variants thereof.

In some embodiments, the naturally occurring variants have at least 95% (e.g., 95-98%), such as at least 98% (e.g., 99-99%), or at least 99% (e.g., 99-100%) homology to a mammalian C4A target nucleic acid, such as a target nucleic acid of SEQ ID NO: 3 and/or SEQ ID NO: 5. In some embodiments, the naturally occurring variants have at least 99% (e.g., 99-100%) homology to the human C4A target nucleic acid of SEQ ID NO: 3. In some embodiments, the naturally occurring variants have at least 95% (e.g., 95-98%), such as at least 98% (e.g., 98-99%), or at least 99% (e.g., 99-100%) homology to a mammalian C4B target nucleic acid, such as a target nucleic acid of SEQ ID NO: 4 and/or SEQ ID NO: 5. In some embodiments, the naturally occurring variants have at least 99% (e.g., 99-100%) homology to the human C4B target nucleic acid of SEQ ID NO: 4. In some embodiments, the naturally occurring variants are known polymorphisms.

Inhibition of Expression

The term “inhibition of expression” as used herein is to be understood as an overall term for a C4 inhibitor's ability to inhibit an amount or the activity of C4 in a target cell. Inhibition of expression or activity may be determined by measuring the level of C4 pre-mRNA or C4 mRNA, or by measuring the level of C4 protein or activity in a cell. Inhibition of expression may be determined in vitro or in vivo. Inhibition is determined by reference to a control. It is generally understood that the control is an individual or target cell treated with a saline composition. In some embodiments, C4 is C4A and/or C4B.

The term “inhibitor,” “inhibition” or “inhibit” may also be referred to as down-regulate, reduce, suppress, lessen, lower, or decrease the amount, expression, and/or activity of C4.

The inhibition of expression of C4A and/or C4B may occur e.g. by degradation of pre-mRNA or mRNA e.g. using RNase H recruiting oligonucleotides, such as gapmers, or nucleic acid molecules that function via the RNA interference pathway, such as siRNA or shRNA. Alternatively, the inhibitor of the present invention may bind to C4A and/or C4B mRNA or polypeptide and inhibit the activity of C4A and/or C4B or prevent its binding to other molecules.

In some embodiments, the inhibition of expression of the C4A and/or C4B target nucleic acid results in a decreased amount of C4A and/or C4B protein in the target cell. Preferably, the amount of C4A and/or C4B protein is decreased as compared to a control. In some embodiments, the decrease in amount of C4A and/or C4B protein is at least 20%, at least 30%, as compared to a control. In some embodiments, the amount of C4A and/or C4B protein in the target cell is reduced by at least 50%, e.g., 50-60%, or at least 60%, e.g., 60-70%, or at least 70%, e.g., 70-80%, at least 80%, e.g., 80-90%, or at least 90%, e.g., 90-95%, when compared to a control.

Sugar Modifications

The oligonucleotide of the invention may comprise one or more nucleosides, which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar-modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering one or more substituent groups on the ribose ring to groups other than hydrogen, or the 2′-OH group naturally found in DNA and RNA nucleosides. Substituents may, for example, be introduced at the 2′, 3′, 4′ or 5′ positions.

High Affinity Modified Nucleosides

A “high affinity modified nucleoside” is a modified nucleotide which, when incorporated into the oligonucleotide, enhances the affinity of the oligonucleotide for its complementary target, for example as measured by the melting temperature (Tm). A high affinity modified nucleoside of the present invention preferably results in an increase in melting temperature in the range of +0.5 to +12 C, more preferably in the range of +1.5 to +10° C. and most preferably in the range of +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

2′ Sugar Modified Nucleosides

A 2′ sugar modified nucleoside is a nucleoside which has a substituent other than H or —OH at the 2′ position (2′ substituted nucleoside) or comprises a 2′ linked biradical capable of forming a bridge between the 2′ carbon and a second carbon in the ribose ring, such as LNA (2′-4′ biradical bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

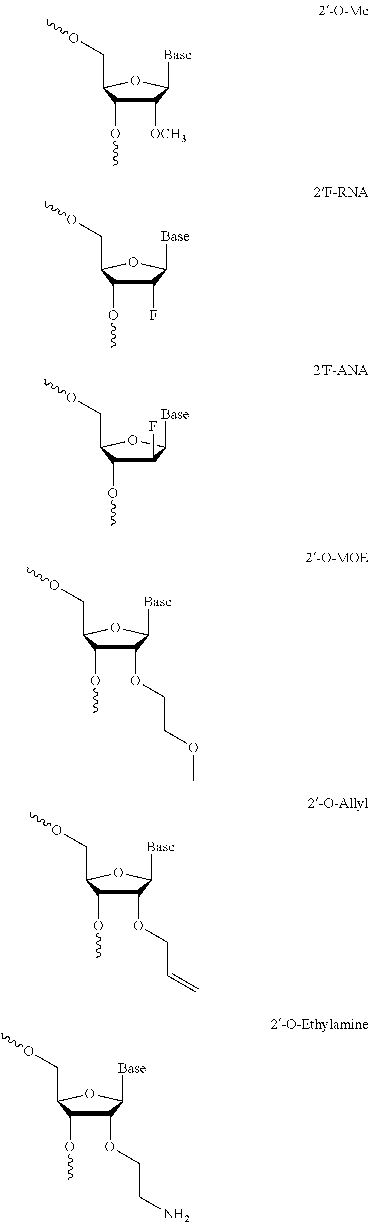

In relation to the present invention, a 2′ substituted sugar modified nucleoside does not include 2′ bridged nucleosides like LNA.

Locked Nucleic Acid Nucleosides (LNA Nucleoside)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acids or bicyclic nucleic acids (BNAs) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Particular examples of LNA nucleosides of the invention are presented in Scheme 1 (wherein B is as defined above).

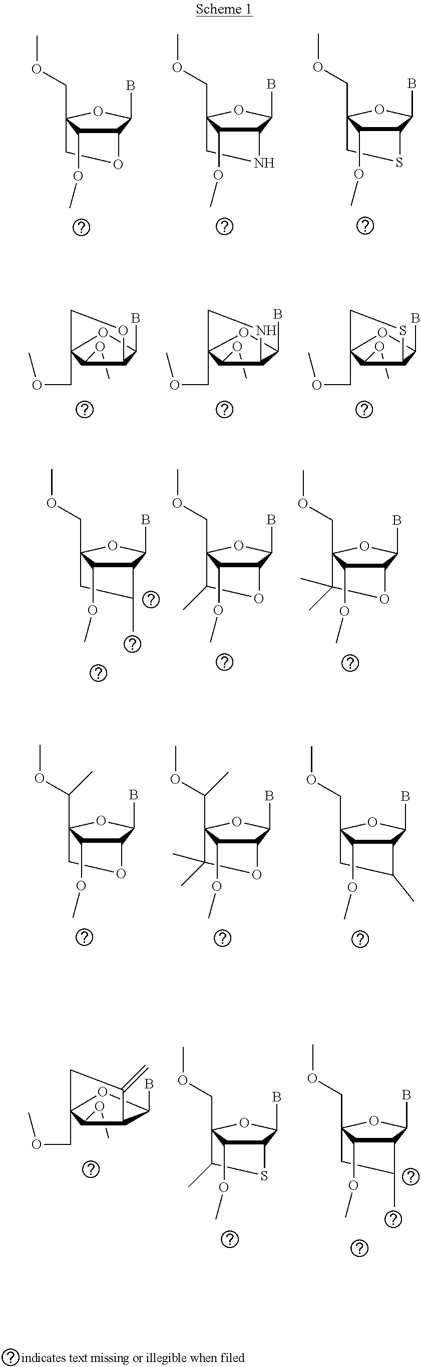

Particular LNA nucleosides for use in molecules of the invention are beta-D-oxy-LNA, 6′-methyl-beta-D-oxy LNA such as (S)-6′-methyl-beta-D-oxy-LNA (ScET) and ENA. A particularly advantageous LNA is beta-D-oxy-LNA.

RNase H Activity and Recruitment

The RNase H activity of an antisense oligonucleotide refers to its ability to recruit RNase H when in a duplex with a complementary RNA molecule. WO01/23613, for example, provides in vitro methods for determining RNase H activity, which may be used to determine ability to recruit RNase H. Typically, an oligonucleotide is deemed capable of recruiting RNase H if it, when provided with a complementary target nucleic acid sequence, has an initial rate, as measured in pmol/l/min, of at least 5%, such as at least 10%-15% or more than 20%, e.g., 20-25%, or 20-30%, of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO 01/23613 (hereby incorporated by reference). For use in determining RNase H activity, recombinant human RNase H1 is available from Creative Biomart® (Recombinant Human RNase H1 fused with His tag expressed in E. coli).

Gapmer

The antisense oligonucleotide of the invention, or contiguous nucleotide sequence thereof, may be a gapmer, also termed gapmer oligonucleotide or gapmer designs. Antisense gapmers are commonly used to inhibit a target nucleic acid via RNase H mediated degradation. A gapmer oligonucleotide comprises at least three distinct structural regions: a 5′-flank, a gap, and a 3′-flank, F-G-F′ in the ‘5->3’ orientation. The “gap” region (G) comprises a stretch of contiguous DNA nucleotides, which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) and/or 3′ (F′) region respectively. The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, such as 15 to 21 nucleosides.

By way of example, the gapmer oligonucleotide of the present invention can be represented by the following formulae:

F₁₋₈-G₅₋₁₆-F′₁₋₈,such as

F₁₋₈-G₇₋₁₆-F′₂₋₈

with the proviso that the overall length of the gapmer regions F-G-F′ is at least 12 (e.g., 12-15 nucleotides), such as at least 14 nucleotides (e.g., 14-20 nucleotides) in length.

In an aspect of the invention, the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and define the 5′ and 3′ ends of the F and F′ region, respectively, and G is a region between 6 and 16 nucleosides which are capable of recruiting RNaseH.

In an aspect of the invention, the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and define the 5′ and 3′ end of the F and F′ region, respectively, and G is a region between 6 and 18 nucleosides which are capable of recruiting RNase H. In some embodiments, the G region consists of DNA nucleosides.

In some embodiments, region F and F′ independently consists of or comprises a contiguous sequence of sugar-modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′-substituted sugar modified nucleotide (mixed wing design). In some embodiments, the 2′-substituted sugar modified nucleotide is independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides. In some embodiments, all the modified nucleosides of region F and F′ are beta-D-oxy LNA nucleosides, wherein region F or F′, or F and F′ may optionally comprise DNA nucleosides. In such embodiments, the flanking region F or F′, or both F and F′ comprise at least three nucleosides, wherein the 5′ and 3′ most nucleosides of the F and/or F′ region are LNA nucleosides.

LNA Gapmer

An “LNA gapmer” is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments, the LNA gapmer is of formula: [LNA]₁₋₅-[region G]₆₋₁₈-[LNA]₁₋₅, wherein region G is as defined in the Gapmer region G definition.

MOE Gapmers

An “MOE gapmer” is a gapmer wherein regions F and F′ consist of MOE (methoxyethy) nucleosides. In some embodiments, the MOE gapmer is of design [MOE]₁₋₈-[Region G]₅₋₁₆-[MOE]₁₋₈, such as [MOE]₂₋₇-[Region G]₆₋₁₄-[MOE]₂₋₇, such as [MOE]₃₋₆-[Region G]₈₋₁₂-[MOE]₃₋₆, such as [MOE]₅-[Region G]₁₀-[MOE]₅ wherein region G is as defined in the Gapmer definition. MOE gapmers with a 5-10-5 design (MOE-DNA-MOE) have been widely used in the art.

Region D′ or D″ in an Oligonucleotide

The oligonucleotide of the invention may in some embodiments comprise or consist of the contiguous nucleotide sequence of the oligonucleotide which is complementary to the target nucleic acid, such as a gapmer region F-G-F′, and may further comprise 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ and D″ can be attached to the 5′ end of region F or the 3′ end of region F′, respectively, to generate designs of the following formulas D′-F-G-F′, F-G-F′-D″ or D′-F-G-F′-D″. In this instance, the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. In some embodiment, the nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments, the additional 5′ and/or 3′ end nucleotides are linked with phosphodiester linkages, and are DNA or RNA. Nucleotide based biocleavable linkers suitable for use as region D′ or D″ are disclosed, for example, in WO2014/076195, which include by way of example a phosphodiester linked DNA dinucleotide. The use of biocleavable linkers in poly-oligonucleotide constructs is disclosed, for example, in WO2015/113922, where they are used to link multiple antisense constructs (e.g. gapmer regions) within a single oligonucleotide.

In one embodiment, the oligonucleotide of the invention comprises a region D′ and/or D″ in addition to the contiguous nucleotide sequence which constitutes the gapmer.

In some embodiments, the oligonucleotide of the present invention can be represented by one or more of the following formulae:

F-G-F′; in particular F₁₋₈-G₅₋₁₈-F′₂₋₈

D′-F-G-F′, in particular D′₁₋₃-F₁₋₈-G₅₋₁₈-F′₂₋₈

F-G-F′-D″, in particular F₁₋₈-G₅₋₁₈-F′₂₋₈-D″₁₋₃

D′-F-G-F′-D″, in particular D′₁₋₃-F₁₋₈-G₅₋₁₈-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Treatment

The term “treatment” as used herein refers to both treatment of an existing disease (e.g. a disease or disorder as herein referred to), or prevention of a disease, i.e. prophylaxis. Prophylaxis also includes delaying or reducing the likelihood of disease occurrence, delaying or reducing frequency of relapse of the disease, and/or reducing severity or duration of the disease if the subject eventually succumbs to the disease. It will therefore be recognized that treatment as referred to herein may, in some embodiments, be prophylactic. In some embodiments, treatment is performed on a patient who has been diagnosed with a complement mediated neurological disease, such as a neurological disease selected from the group consisting of Alzheimer's disease, frontotemporal dementia, multiple sclerosis, amyotrophic lateral sclerosis, Huntington's disease, Parkinson's disease, virus-induced cognitive impairment, glaucoma, macular degeneration, myasthenia gravis, Guillain-Barré syndrome, neuromyelitis optica, central nervous system lupus erythematosus, and schizophrenia. In some embodiments, the compounds of the invention are for use in the treatment of a tauopathy, such as Alzheimer's disease. In some embodiments, the compounds of the invention are for use in the treatment of schizophrenia.

Patient

For the purposes of the present invention, the “subject” (or “patient”) may be a vertebrate. In context of the present invention, the term “subject” includes both humans and other animals, particularly mammals, and other organisms. Thus, the herein provided means and methods are applicable to both human therapy and veterinary applications. Preferably, the subject is a mammal. More preferably, the subject is human.

As described elsewhere herein, the patient to be treated may suffer from or be susceptible to a neurological disease or neurodegenerative disorder. A patient “susceptible to” a disease or disorder is one who is pre-disposed thereto and/or otherwise at risk of developing or having a recurrence of the disease or disorder. A susceptible patient can be understood a patent likely to develop the disease or disorder, to the extent that the patient would benefit from prophylactic treatment or intervention.

By “neurological disease” is meant a disease or disorder of the nervous system including, but not limited to, neurological conditions associated with cancer, and neurodegenerative disease.

By “neurodegenerative disease” is meant diseases including, but not limited to Alzheimer's disease, frontotemporal dementia, multiple sclerosis, amyotrophic lateral sclerosis, Huntington's disease, Parkinson's disease, virus-induced cognitive impairment, glaucoma, macular degeneration, myasthenia gravis, Guillain-Barré syndrome, neuromyelitis optica, central nervous system lupus erythematosus, and schizophrenia. In some embodiments, the patient to be treated suffers from a tauopathy, such as Alzheimer's disease. In some embodiments, the patient to be treated suffers from schizophrenia.

Alzheimer's disease (AD), also referred to as Alzheimer disease or “Alzheimer's,” is a chronic neurodegenerative disorder typically characterized by progressive cognitive deterioration, as well as increasing memory loss, problems with language, judgment, and/or problem solving, and that can lead to inability to perform daily tasks, and eventually dementia.

### DETAILED DESCRIPTION OF THE INVENTION

Synapse removal and neuronal damage can be mediated by the classical pathway of the complement system, which is initiated by activation of the C1 complex (consisting of C1Q, C1S and C1R), leading to cleavage of C2 and C4, which in turn lead to cleavage of C3 which can trigger phagocytosis as well as inflammation and further downstream complement activation. In the context of the present invention, the present inventors have shown that nucleic acid molecules, such as antisense oligonucleotides, inhibit the expression of C4A and/or C4B. Reduced expression of C4 can lead to reduced cleavage of C3 and thereby to reduced engulfment of synapses by microglia cells and other harmful effects of complement activation.

One aspect of the present invention is a C4 inhibitor for use in the treatment and/or prevention of a neurological disease, in particular a neurological disease selected from a tauopathy and schizophrenia. In some embodiments, the tauopathy is Alzheimer's disease. The C4 inhibitor can for example be a small molecule that specifically binds to a C4 protein, wherein said inhibitor prevents or reduces cleavage of the C4 protein.

An embodiment of the invention is a C4 inhibitor, which is capable of preventing or reducing expression of C4A protein and/or C4B protein thereby leading to reduced cleavage of C3. In some embodiments, the C4 inhibitor leads to inhibition of engulfment of synapses by microglia cells.

C4 Inhibitors for Use in Treatment of Neurological Diseases

Without being bound by theory, it is believed that C4 is involved in the in the cleavage of C3 and thereby in the engulfment of synapses by microglia cells.

In some embodiments of the present invention, the inhibitor is small molecule compound. In some embodiments, the inhibitor may be a small molecule that specifically binds to the C4A and/or C4B protein. In some embodiments, the C4A protein is encoded by a sequence selected from SEQ ID NO: 3, 5, and 6. In some embodiments, the C4B protein is encoded by a sequence selected from SEQ ID NO: 4, 5, and 7.

Nucleic Acid Molecules of the Invention

Therapeutic nucleic acid molecules find use as C4 inhibitors since they can target C4 transcripts and promote their degradation, e.g., either via the RNA interference pathway or via RNase H cleavage. Alternatively, oligonucleotides such as aptamers can also act as inhibitors of C4 proteins.

One aspect of the present invention is a C4 targeting nucleic acid molecule for use in treatment and/or prevention of Neurological diseases. Such a nucleic acid molecule can be selected from the group consisting of a single stranded antisense oligonucleotide, an siRNA, and a shRNA.

The present section describes novel nucleic acid molecules suitable for use in treatment and/or prevention of a neurological disease. In some embodiments, the neurological disease is selected from the group consisting of Alzheimer's disease, frontotemporal dementia, multiple sclerosis, amyotrophic lateral sclerosis, Huntington's disease, Parkinson's disease, virus-induced cognitive impairment, glaucoma, macular degeneration, myasthenia gravis, Guillain-Barre syndrome, neuromyelitis optica, central nervous system lupus erythematosus, and schizophrenia. In some embodiments, the neurological disease is a tauopathy, such as Alzheimer's disease. In some embodiments, the neurological disease is schizophrenia.

The nucleic acid molecules of the present invention are capable of inhibiting C4 mRNA and/or expression of C4 protein in vitro and in vivo. The inhibition can be achieved by hybridizing an oligonucleotide to a target nucleic acid encoding a C4A and/or C4B protein. In some embodiments, the target nucleic acid may be a mammalian C4A sequence. In some embodiments, the target nucleic acid may be a human C4A pre-mRNA sequence such as the sequence of SEQ ID NO: 3 or a human mature C4A mRNA sequence such as the sequence of SEQ ID NO: 6. In some embodiments, the target nucleic acid may be a mammalian C4B sequence. In some embodiments, the target nucleic acid may be a human C4B pre-mRNA sequence such as the sequence of SEQ ID NO: 4 or a human mature C4B mRNA sequence such as the sequence of SEQ ID NO: 7. In some embodiments, the target nucleic acid may be a cynomolgus monkey C4 sequence such as the sequence of SEQ ID NO: 5.

In some embodiments, the nucleic acid molecule of the invention is capable of modulating the expression of the target by inhibiting or down-regulating it. Preferably, such modulation produces an inhibition of expression of at least 20% (e.g., 20-30%) compared to the normal expression level of the target, more preferably at least 30% (e.g., 30-40%), at least 40% (e.g., 40-50%), or at least 50% (e.g., 50-60%), inhibition compared to the normal expression level of the target. In some embodiments, the nucleic acid molecule of the invention may be capable of inhibiting expression levels of C4 mRNA by at least 50% (e.g., 50-60%) or 60% (e.g., 50-60%) in vitro by using 20-50 nM nucleic acid molecule for transfection. In some embodiments, the nucleic acid molecule of the invention may be capable of inhibiting expression levels of C4 mRNA by at least 50% (e.g., 50-60%) or 60% (e.g., 50-60%) in vitro by using 50-350 nM nucleic acid molecule for gymnosis. Suitably, the examples provide assays, which may be used to measure C4 mRNA inhibition (e.g. Example 1 and the “Materials and Methods” section). C4 inhibition is triggered by the hybridization between a contiguous nucleotide sequence of the oligonucleotide, such as the guide strand of a siRNA or gapmer region of an antisense oligonucleotide, and the target nucleic acid. In some embodiments, the nucleic acid molecule of the invention comprises mismatches between the oligonucleotide and the target nucleic acid. Despite mismatches, hybridization to the target nucleic acid may still be sufficient to show a desired inhibition of C4 expression. Reduced binding affinity resulting from mismatches may advantageously be compensated by increased number of nucleotides in the oligonucleotide complementary to the target nucleic acid and/or an increased number of modified nucleosides capable of increasing the binding affinity to the target, such as 2′ sugar modified nucleosides, including LNA, present within the oligonucleotide sequence.

An aspect of the present invention relates to a nucleic acid molecule of 12 to 60 nucleotides in length, which comprises a contiguous nucleotide sequence of at least 12 nucleotides in length, such as at least 12 to 30 nucleotides in length, which is at least 95% complementary, such as fully complementary, to a mammalian C4 target nucleic acid, in particular a human C4 mRNA. These nucleic acid molecules are capable of inhibiting the expression of C4 mRNA and/or C4 protein.

An aspect of the invention relates to a nucleic acid molecule of 12 to 30 nucleotides in length, comprising a contiguous nucleotide sequence of at least 12 nucleotides, such as 12 to 30, or such as 15 to 21 nucleotides in length, which is at least 90% complementary, such as fully complementary, to a mammalian C4 target sequence.

A further aspect of the present invention relates to a nucleic acid molecule according to the invention comprising a contiguous nucleotide sequence of 14 to 22, such as 15 to 21 nucleotides in length with at least 90% complementary, such as fully complementary, to the target sequence of SEQ ID NO: 3 and/or 4.

In some embodiments, the nucleic acid molecule comprises a contiguous sequence of 12 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

It is advantageous if the oligonucleotide, or contiguous nucleotide sequence thereof, is fully complementary (100% complementary) to a region of the target sequence, or in some embodiments may comprise one or two mismatches between the oligonucleotide and the target sequence.

In some embodiments, the oligonucleotide sequence is 100% complementary to a region of the target sequence of SEQ ID NO: 3 and/or 4. In some embodiments, the oligonucleotide sequence is 100% complementary to a region of the target sequence of SEQ ID NO: 6 and/or 7.

In some embodiments, the nucleic acid molecule or the contiguous nucleotide sequence of the invention is at least 90% or 95% complementary, such as fully (or 100%) complementary, to the target nucleic acid of SEQ ID NO: 3 and/or 4.

In some embodiments, the oligonucleotide or the contiguous nucleotide sequence of the invention is at least 90% or 95% complementary, such as fully (or 100%) complementary, to the target nucleic acid of SEQ ID NO: 5 and/or SEQ ID NO: 6 and 7.

In some embodiments, the oligonucleotide or the contiguous nucleotide sequence of the invention is at least 90% or 95% complementary, such as fully (or 100%) complementary, to the target nucleic acid of SEQ ID NO: 1 and 2, and/or SEQ ID NO: 3 and 4, and/or SEQ ID NO: 5.

In some embodiments, the contiguous sequence of the nucleic acid molecule of the present invention is least 90% complementary, such as fully complementary to a region of SEQ ID NO: 3 and/or 4, selected from the group consisting of target regions 1A to 297A as shown in Table 4a and/or regions 1B to 297B as shown in Table 4b.

In some embodiments, the nucleic acid molecule of the invention comprises or consists of 12 to 60 nucleotides in length, such as from 13 to 50, such as from 14 to 35, such as 15 to 30, such as from 15 to 21 contiguous nucleotides in length. In a preferred embodiment, the nucleic acid molecule comprises or consists of 15, 16, 17, 18, 19, 20 or 21 nucleotides in length.

In some embodiments, the contiguous nucleotide sequence of the nucleic acid molecule, which is complementary to the target nucleic acids, comprises or consists of 12 to 30, such as from 13 to 25, such as from 15 to 21 contiguous nucleotides in length.

In some embodiments, the oligonucleotide is selected from the group consisting of an antisense oligonucleotide, an siRNA and a shRNA.

In some embodiments, the contiguous nucleotide sequence of the siRNA or shRNA, which is complementary to the target sequence, comprises or consists of 18 to 28, such as from 19 to 26, such as from 20 to 24, such as from 21 to 23, contiguous nucleotides in length.

In some embodiments, the contiguous nucleotide sequence of the antisense oligonucleotide, which is complementary to the target nucleic acids, comprises or consists of 12 to 22, such as from 14 to 21, such as from 15 to 21 such as from 15, 16, 17, 18, 19, 20, or 21 contiguous nucleotides in length.

In some embodiments, the oligonucleotide or contiguous nucleotide sequence comprises or consists of a sequence selected from the group consisting of sequences listed in Table 7.

It is understood that the contiguous oligonucleotide sequence (motif sequence) can be modified to, for example, increase nuclease resistance and/or binding affinity to the target nucleic acid.

The pattern in which the modified nucleosides (such as high affinity modified nucleosides) are incorporated into the oligonucleotide sequence is generally termed oligonucleotide design.

The nucleic acid molecule of the invention may be designed with modified nucleosides and RNA nucleosides (in particular for siRNA and shRNA molecules) or DNA nucleosides (in particular for single stranded antisense oligonucleotides).

In advantageous embodiments, the nucleic acid molecule or contiguous nucleotide sequence comprises one or more sugar modified nucleosides, such as 2′ sugar modified nucleosides, such as comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In some embodiments, the contiguous nucleotide sequence comprises LNA nucleosides.

In some embodiments, the contiguous nucleotide sequence comprises LNA nucleosides and DNA nucleosides.

In some embodiments, the contiguous nucleotide sequence comprises 2′-O-methoxyethyl (2′MOE) nucleosides.

In some embodiments, the contiguous nucleotide sequence comprises 2′-O-methoxyethyl (2′MOE) nucleosides and DNA nucleosides.

Advantageously, the 3′ most nucleoside of the antisense oligonucleotide, or contiguous nucleotide sequence thereof, is a 2′sugar modified nucleoside.

In a further embodiment, the nucleic acid molecule comprises at least one modified internucleoside linkage. Suitable internucleoside modifications are described in the “Definitions” section under “Modified internucleoside linkage”.

Advantageously, the oligonucleotide comprises at least one modified internucleoside linkage, such as phosphorothioate or phosphorodithioate.

In some embodiments, at least one internucleoside linkage in the contiguous nucleotide sequence is a phosphodiester internucleoside linkage.

It is advantageous if at least 2 to 3 internucleoside linkages at the 5′ or 3′ end of the oligonucleotide are phosphorothioate internucleoside linkages.

For single stranded antisense oligonucleotides, it is advantageous if at least 75%, such as 70-80%, at least 90%, such as 90-95%, or all, the internucleoside linkages within the contiguous nucleotide sequence are phosphorothioate internucleoside linkages. In some embodiments, all the internucleotide linkages in the contiguous sequence of the single stranded antisense oligonucleotide are phosphorothioate linkages.

In an advantageous embodiment of the invention, the antisense oligonucleotide of the invention is capable of recruiting RNase H, such as RNase H1. An advantageous structural design is a gapmer design as described in the “Definitions” section under for example “Gapmer”, “LNA Gapmer” and

“MOE gapmer”. In the present invention, it is advantageous if the antisense oligonucleotide of the invention is a gapmer with an F-G-F′ design.

In some embodiments, the F-G-F′ design may further include region D′ and/or D″ as described in the “Definitions” section under “Region D′ or D” in an oligonucleotide”.

In some embodiments, the inhibitor of the present invention is a nucleic acid capable of inducing the process of RNA interference (as described, e.g., in WO 2014/089121).

Method of Manufacture

In a further aspect, the invention provides methods for manufacturing the oligonucleotide of the invention. In some embodiments, the method comprises reacting nucleotide units and thereby forming covalently linked contiguous nucleotide units comprised in the oligonucleotide in a sequence according to a nucleic acid molecule of the present invention. Preferably, the method uses phophoramidite chemistry (see for example Caruthers et al, 1987, Methods in Enzymology vol. 154, pages 287-313).

The manufactured oligonucleotides may comprise one or more modifications as described herein. For example, the manufactured oligonucleotides may comprise one or more sugar-modified nucleosides, one or more modified internucleoside linkages and/or one or more modified nucleobases. Accordingly, the method for manufacturing the oligonucleotide of the invention may further comprise the introduction of such modifications into the oligonucleotide.

In some embodiments, one or more modified internucleoside linkages, such as phosphorothioate internucleoside linkages, may be introduced into the oligonucleotide. In some embodiments, one or more sugar-modified nucleosides, such as 2′ sugar modified nucleosides, may be introduced. In some embodiments, one or more high affinity modified nucleosides and/or one or more LNA nucleosides may be introduced into the oligonucleotide. In some embodiments, region D′ and/or D″ as described elsewhere herein are added to the oligonucleotide.

In a further aspect, a method is provided for manufacturing the pharmaceutical composition of the invention, comprising mixing the oligonucleotides of the invention with a pharmaceutically acceptable diluent, solvent, carrier, salt and/or adjuvant.

As described elsewhere herein in more detail, the oligonucleotide of the invention may exist in the form of its pharmaceutically acceptable salts, esters, solvates or in the form of prodrugs. Accordingly, methods are provided for manufacturing the oligonucleotide of the invention in such forms.

Pharmaceutically Salts

The compounds according to the present invention may exist in the form of their pharmaceutically acceptable salts. The term “pharmaceutically acceptable salt” refers to conventional acid-addition salts or base-addition salts that retain, or substantially retain, the biological effectiveness and properties of the compounds of the present invention. By way of example, the following salts may be mentioned: Alkaline metal salts such as sodium salts, potassium salts or lithium salts; alkaline earth metal salts such as calcium salts or magnesium salts: metal salts such as aluminum salts, iron salts, zinc salts, copper salts; amine salts including inorganic salts such as ammonium salts and organic salts such as t-octylamine salts, dibenzylamine salts, morpholine salts, glucosamine salts, phenylglycine alkyl ester salts, ethylenediamine salts, N-methylglucamine salts, guanidine salts, diethylamine salts, triethylamine salts, dicyclohexylamine salts, dibenzylethylenediamine salts, chloroprocaine salts, procaine salts, diethanolamine salts, N-benzyl-phenethylamine salts, piperazine salts, tetramethylammonium salts or tris(hydroxymethyl)aminomethane salts, inorganic acid salts including hydrohalogenic acid salts such as hydrofluorides, hydrochlorides, hydrobromides or hydroiodides, sulfates or phosphates; organic acid salts including lower alkane sulfonic acid salts such as methanesulfonates, trifluoromethanesulfonates or ethanesulfonates, arylsulfonic acid salts such as benzenesulfonates or p-toluenesulfonates, acetates, malates, fumarates, succinates, citrates, tartrates, oxalates or maleates; and amino acid salts such as glycine salts, lysine salts, arginine salts, ornithine salts, glutamic acid salts or aspartic acid salts. These salts may be prepared by known methods.

In a further aspect, the invention provides a pharmaceutically acceptable salt of the nucleic acid molecule of the invention, such as a pharmaceutically acceptable sodium salt, ammonium salt or potassium salt.

Solvates

The compounds according to the present invention may exist in the form of solvates. The term ‘solvate’ is used herein to describe a molecular complex comprising the oligonucleotide of the invention and one or more pharmaceutically acceptable solvent molecules, for example, ethanol or water. If the solvent is water, the solvate is a “hydrate”. Pharmaceutically acceptable solvates within the meaning of the present invention include hydrates and other solvates.

Prodrugs

Further, the compounds according to the present invention may be administered in the form of a prodrug. A prodrug is defined as a compound that undergoes transformations in vivo to yield the parent active drug. Because cell membranes are lipophilic in nature, cellular uptake of oligonucleotides is often reduced compared to neutral or lipophilic equivalents. One solution is to use a prodrug approach (see e.g. Crooke, R. M. (1998) in Crooke, S. T. Antisense research and Application. Springer-Verlag, Berlin, Germany, vol. 131, pp. 103-140). Examples of such prodrugs include, but are not limited to, amides, esters, carbamates, carbonates, ureides and phosphates. These prodrugs may be prepared by known methods.

Pharmaceutical Composition

In a further aspect, the invention provides pharmaceutical compositions comprising any of the compounds of the invention, in particular the aforementioned nucleic acid molecules or salts thereof and a pharmaceutically acceptable diluent, carrier, salt and/or adjuvant. A pharmaceutically acceptable diluent includes, but is not limited to, phosphate-buffered saline (PBS). Pharmaceutically acceptable salts include, but are not limited to, sodium and potassium salts. In some embodiments the pharmaceutically acceptable diluent is sterile phosphate buffered saline. In some embodiments, the nucleic acid molecule is used in the pharmaceutically acceptable diluent at a concentration of 50 to 300 μM solution. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091, e.g., provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations, and the like, are also provided, e.g., in WO2007/031091. In some embodiments, the nucleic acid molecule of the invention, or pharmaceutically acceptable salt thereof is in a solid form, such as a powder, such as a lyophilized powder. Compounds or nucleic acid molecules of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

Administration

The oligonucleotides or pharmaceutical compositions of the present invention may be administered via parenteral (such as, intravenous, subcutaneous, intra-muscular, intranasal, intracerebral, intracerebroventricular intraocular, or intrathecal administration).

In some embodiments, the administration is via intrathecal administration, e.g., by lumbar puncture.

Advantageously, e.g. for treatment of neurological disorders, the oligonucleotide or pharmaceutical compositions of the present invention are administered intrathecally or intracranially, e.g. via intracerebral or intraventricular administration.

The invention also provides for the use of the oligonucleotide or conjugate thereof, such as pharmaceutical salts or compositions of the invention, for the manufacture of a medicament wherein the medicament is in a dosage form for subcutaneous administration.

The invention also provides for the use of the oligonucleotide of the invention, or conjugate thereof, such as pharmaceutical salts or compositions of the invention, for the manufacture of a medicament wherein the medicament is in a dosage form for intrathecal administration.

In some embodiments, a therapeutically or prophylactically effective amount of the oligonucleotide or pharmaceutical composition of the present invention is administered.

Delivery Platforms

Delivery of the oligonucleotides to the target tissue may be enhanced by carrier-mediated delivery including, but not limited to, cationic liposomes, cyclodextrins, porphyrin derivatives, branched chain dendrimers, polyethylenimine polymers, nanoparticles, cell-penetrating peptides, and microspheres (see e.g. Dass, C R. J Pharm Pharmacol 2002; 54(1):3-27).

In some embodiments, the inhibitors of the present invention, such as the oligonucleotides of the present invention, are targeted to the brain. For example, delivery to the brain might be achieved by conjugating said inhibitor to a moiety that facilitates delivery across the blood brain barrier, such as an antibody or antibody fragment targeting the transferrin receptor.

Combination Therapies

In some embodiments, the inhibitor of the present invention such as the nucleic acid molecule, nucleic acid molecule conjugate, pharmaceutically acceptable salt, or pharmaceutical composition of the invention is for use in a combination treatment with another therapeutic agent. The therapeutic agent can for example be the standard of care for the diseases or disorders described above.

By way of example, the inhibitor of the present invention may be used in combination with other actives, such as oligonucleotide-based therapeutic agents—such as sequence specific oligonucleotide-based therapeutic agents—acting through nucleotide sequence-dependent mode of action.

By way of further example, the inhibitor of the present invention may be used in combination with one or more acetylcholinesterase inhibitors and/or one or more NMDA receptor antagonists. A cholinesterase inhibitor may be, for example, donepezil, tacrine, galantamine or rivastigmine. A NMDA receptor antagonist may be, for example, memantine.

By way of further example, the inhibitor of the present invention may be used in combination with one or more typical antipsychotics and/or one or more atypical antipsychotics. A typical antipsychotic may be, for example, chlorpromazine, fluphenazine, haloperidol, perphenazine, thioridazine, thiothixene, or trifluoperazine. An atypical antipsychotic may be, for example, aripiprazole, aripiprazole lauroxil, asenapine, brexpiprazole, cariprazine, clozapine, Iloperidone, lumateperone tosylate, lurasidone, olanzapine, paliperidone, aliperidone palmitate, or ziprasidone.

In some embodiments, the inhibitor of the present invention is used in combination with an antibody that binds to complement C4 or the C4b portion of C4 (e.g., as described in WO 2017/196969).

In some embodiments, the inhibitor of the present invention is used in combination with one or more of the following: an antisense compound that targets C9ORT72 (e.g., as described in WO 2014/062736); an antisense oligonucleotide, aptamer, miRNA, ribozyme, or siRNA that blocks expression of one or more of C3 convertase, C5, C6, C7, C8, and C9 (e.g., as described in WO 2008/044928); an antibody that blocks the activity of one or more of C3 convertase, C5, C6, C7, C8, and C9 (e.g., as described in WO 2008/044928); an antisense or double stranded RNA that decreases activity of the complement cascade (e.g., as described in WO 2005/060667); and an antibody that binds C1s protein, e.g., to inhibit proteolytic activity of C1s (e.g., as described in WO 2014/066744).

In some embodiments, the inhibitor of the present invention is used in combination with one or more nucleic acid molecules disclosed in U.S. Provisional application filed May 11, 2020, entitled “Complement Component C1R Inhibitors For Treating A Neurological Disease, And Related Compositions, Systems And Methods Of Using Same” and US Provisional application filed May 11, 2020, entitled “Complement Component C1S Inhibitors For Treating A Neurological Disease, And Related Compositions, Systems And Methods Of Using Same,”

Applications

The nucleic acid molecules of the invention may be utilized as research reagents for, for example, diagnostics, as well as for therapeutics and prophylaxis.

In research, such nucleic acid molecules may be used to specifically modulate the synthesis of a C4 protein in cells (e.g. in vitro cell cultures) and animal models thereby facilitating functional analysis of the target or an appraisal of its usefulness as a target for therapeutic intervention. Typically, the target modulation is achieved by degrading or inhibiting the mRNA corresponding to the protein, thereby preventing protein formation or by degrading or inhibiting a modulator of the gene or mRNA producing the protein.

If employing the nucleic acid molecules of the invention in research or diagnostics, the target nucleic acid may be a cDNA or a synthetic nucleic acid derived from DNA or RNA.

Methods of Detection or Diagnosis

Further encompassed by the present invention is a method for diagnosing a neurological disease in a patient suspected of a having a neurological disease, said method comprising the step of
a) determining the amount of one or more C4 nucleic acids, such as C4 mRNA or cDNA derived from C4 mRNA, in a sample from the subject, wherein the determination comprises contacting the sample with one or more oligonucleotides of the present invention,
b) comparing the amount determined in step a) to a reference amount, and
c) diagnosing whether the subject suffers from the neurological disease, or not, based on the results of step b).

In some embodiments, the method of diagnosing a neurological disease is an in vitro method.

The term “neurological disease” has been defined elsewhere herein. The definition applies accordingly. In some embodiments, the neurological disease to be diagnosed is a tauopathy, such as Alzheimer's disease. In some embodiments, the neurological disease to be diagnosed is schizophrenia.

The term “sample” refers to a sample of a body fluid, to a sample of separated cells or to a sample from a tissue or an organ. Samples of body fluids can be obtained by well-known techniques and include samples of blood, plasma, serum, urine, lymphatic fluid, sputum, ascites, saliva, and lacrimal fluid. In some embodiments, the sample is a cerebrospinal fluid sample.

Tissue or organ samples may be obtained from any tissue or organ by, e.g., biopsy. In some embodiments, the sample is a neural tissue sample, such as a brain tissue sample or spinal cord sample.

In some embodiments, the sample comprises neuron, astrocytes, oligodendrocytes, and/or microglia cells.

The subject may be a mammal. In some embodiments, the subject is a human. In some embodiments, the subject is a human. In some embodiments, the subject is a cynomolgus monkey.

In step a) of the aforementioned method, the amount of C4 nucleic acid present in the sample shall be determined. The C4 nucleic acid to be determined shall be a nucleic acid encoding a C4 protein, such as a C4A or C4B protein. In some embodiments, the C4 nucleic acid is mammalian C4 nucleic acid. In some embodiments, the C4 nucleic acid is a human C4 nucleic acid, such as a human C4A or C4B nucleic acid.

The C4 nucleic acid may for example be a gene, a RNA, a mRNA, and pre-mRNA, a mature mRNA or a cDNA sequence. In an embodiment, the nucleic acid is a C4 mRNA, such as a C4A or C4B mRNA. In another embodiment, the C4 nucleic acid is cDNA derived from a C4 mRNA.

In step b) of aforementioned method, the amount of the C4 nucleic acid shall be compared to a reference, i.e. to a reference amount. The terms “reference amount” or “reference” are well understood by the skilled person. Suitable reference amounts can, in principle, be calculated for a cohort of subjects based on the average or mean values for a given biomarker by applying standard methods of statistics. A suitable reference shall allow for the diagnosis of the neurological disease. Accordingly, the reference shall allow for differentiating between a patient suffering from a neurological disease and a subject who is not suffering from a neurological disease. In some embodiments, the reference is a predetermined value.

In some embodiments, an amount of the one or more C4 nucleic acids larger than the reference amount is indicative for a patient suffering from a neurological disease, whereas an amount of the one or more C4 nucleic acids lower than the reference amount is indicative for a patient not suffering from neurological disease.

The determination of the amount of the one or more nucleic acids in step a) shall comprise contacting the sample with one or more oligonucleotides of the present invention. For example, the sample is contacted with said one or more oligonucleotides under conditions, which allow for the hybridization of said one or more oligonucleotides to the one or more C4 nucleic acids present in the sample (such as the C4 mRNA), thereby forming duplexes of said oligonucleotides and said C4 nucleic acids. In some embodiments, the amount of the one or more C4 nucleic acids is determined by determining the amount of the formed duplexes, e.g. via a detectable label. Accordingly, the one or more oligonucleotides to be used may comprise a detectable label.

Further encompassed by the present invention is a method for detecting one or more C4 nucleic acids in a sample, for example, in a sample as defined above. The method may comprise contacting the sample with one or more oligonucleotides of the present invention as described above. In some embodiments, the sample is from a patient having or suspected of a having a neurological disease.

Also encompassed by the present invention is an in vivo or in vitro method for modulating C4 expression in a target cell, which is expressing C4, said method comprising administering a nucleic acid molecule, conjugate compound, or pharmaceutical composition of the invention in an effective amount to said cell.

In some embodiments, the target cell is a mammalian cell, in particular a human cell. The target cell may be an in vitro cell culture or an in vivo cell forming part of a tissue in a mammal. In preferred embodiments, the target cell is present in the brain. The target cell may be a brain cell. In some embodiments, the brain cell is selected from the group consisting of a neuron, an astrocyte, an oligodendrocyte, and a microglia cell.

One aspect of the present invention is related to the nucleic acid molecules or pharmaceutical compositions of the invention for use as a medicament.

In an aspect of the invention, the C4 inhibitor, such as a nucleic acid molecule or pharmaceutical composition of the invention, is capable of reducing the amount of C4 in a cell expressing C4.

For example, a nucleic acid molecule that inhibits C4 expression may reduce the C4 protein in an affected cell by at least 50% (e.g., 50-60%), or at least 60% (e.g., 60-70%), or at least 70% (e.g., 70-80%), at least 80% (e.g., 80-90%), or at least 90% (e.g., 90-95%) reduction compared to controls. The controls may be untreated cells or animals, or cells or animals treated with an appropriate control.

Inhibition of C4 expression may be measured by RT-qPCR, e.g. as described in the Materials and Methods section.

Due to the decrease of C4 levels, the nucleic acid molecules or pharmaceutical compositions of the present invention can be used to inhibit development of or in the treatment of Neurological diseases.

Accordingly, one aspect of the present invention is related to use of an C4 inhibitor, such as the nucleic acid molecule or pharmaceutical compositions of the invention to decrease C4 protein in an individual having or susceptible to a neurological disease.

The subject to be treated with the C4 inhibitor, such as the nucleic acid molecules or pharmaceutical compositions of the invention (or who prophylactically receives nucleic acid molecules or pharmaceutical compositions of the present invention) is preferably a human, more preferably a human patient who has a neurological diseases, even more preferably a human patient having a tauopathy, even more preferably a human patient having Alzheimer's disease. In some embodiments, the human patient has schizophrenia.

Accordingly, the present invention relates to a method of treating Neurological diseases, wherein the method comprises administering an effective amount of a C4 inhibitor, such as a nucleic acid molecule or pharmaceutical composition of the invention. The present invention further relates to a method of preventing Neurological diseases. In one embodiment, the C4 inhibitors of the present invention is not intended for the treatment of Neurological diseases, only its prevention.

The invention also provides for the use of a C4 inhibitor, such as nucleic acid molecule or a pharmaceutical composition of the invention, for the manufacture of a medicament, in particular a medicament for use in the treatment of Neurological diseases. In preferred embodiments, the medicament is manufactured in a dosage form for intrathecal or intracranial administration.

In some embodiments, the subject to be treated does not have a cardiovascular disorder or disease (e.g., as described in WO 2014/089121). In some embodiments, the subject to be treated does not require treatment for pain (e.g., as described in WO 2005/060667).

The invention also provides for the use of the nucleic acid molecule or the pharmaceutical composition of the invention for the manufacture of a medicament wherein the medicament is in a dosage form for intravenous administration.

In some embodiments, C4 is C4A and/or C4B.

Kits

The invention also provides a kit containing the C4 inhibitor of the present invention, such as the nucleic acid molecule or pharmaceutical composition of the present invention, and instructions for administering the C4 inhibitor. The instructions may indicate that the C4 inhibitor may be used for the treatment of a neurological disease or neurodegenerative disorder as referred to herein, such as Alzheimer's disease or Schizophrenia.

The term “kit” as used herein refers to a packaged product comprising components with which to administer the C4 inhibitor of the present invention. The kit may comprise a box or container that holds the components of the kit. The kit can also include instructions for administering the C4 inhibitor of the present invention of the invention.

### EXAMPLES

Materials and Methods

### Example 1: Testing In Vitro Efficacy of Antisense Oligonucleotides Targeting C4 in Primary Mouse Hepatocytes

Cells were maintained in a humidified incubator as recommended by the supplier. The vendor and recommended culture conditions are reported in Table 5.

TABLE 5
Cell culture details.

| | | | Seeding | Incub. time | Incub. |
|---|---|---|---|---|---|
| | | | density | before oligo | time with |
| Cell Line | Vendor | Culture Condition | (cells/well) | (hrs) | oligo (hrs) |
| mouse | Minerva | WME (Sigma #W1878) w/FBS: | 25000 | 24 | 72 |
| hepatocytes | Imaging | complemented with 1x | | | |
| | | Pen/Strep/Glutamine (freshly | | | |
| | | added), 10% (v/v) FBS (Sigma | | | |
| | | F7524), non-heat inactivated. | | | |

For assays, cells were seeded in a 96-multi well plate in culture media and incubated as reported in Table 5 before addition of oligonucleotides dissolved in PBS. The seeding density of the cells is reported in Table 5.

Oligonucleotides were added at the concentrations reported in Table 8. The cells were harvested 72 hours after the addition of oligonucleotides (see Table 5). RNA was extracted using the RNeasy 96 kit (Qiagen) according to the manufacturer's instructions and eluted in 200 μL of water. The RNA was subsequently heated to 90° C. for one minute.

For gene expressions analysis, One Step RT-qPCR was performed using gScript™ XLT One-Step RT-qPCR ToughMix®, Low ROX™ (Quantabio) in a duplex set up. The primer assays used for qPCR are collated in Table 6 for both target and endogenous control.

TABLE 6
qPCR primer-probe details.

| | | Endogen. | | | |
|---|---|---|---|---|---|
| Endogen | Endog. contr. | contr. | | | Target |
| contr. assay | vendor | fluorophore | Target assay | Target vendor | fluorophore |
| RPLP0: | IDT | HEX-ZEN | C4: | Thermo | FAM-MGB |
| Rplp0_MmPT5843894205 | | | C4_Mm00550309_m1 | Scientific | |
| RPLP0: | IDT | HEX-ZEN | C4B: | Thermo | FAM-MGB |
| Rplp0_MmPT5843894205 | | | C4B_Mm00437890_m1 | Scientific | |

Provided herein are the following oligonucleotide compounds (Table 7):

TABLE 7
Oligonucleotide compounds

| | | | SEQ | | | | | |
|---|---|---|---|---|---|---|---|---|
| SEQ | | | ID | | CMP | | start_ | start_ |
| NO | Motif | Design | NO: | Compound | ID NO | ΔG° | C4a | C4b |
| 42 | CCAAATTAACCACAGAA | 4−10−3 | 303 | CCAAattaaccacaGAA | 264_1 | −19.8 | 243 | 240 |
| 43 | GTCCCCAAATTAACCACA | 2−14−2 | 304 | GTccccaaattaaccaCA | 265_1 | −22.9 | 246 | 243 |
| 44 | GTCCCCAAATTAACCAC | 2−12−3 | 305 | GTccccaaattaacCAC | 266_1 | −22.1 | 247 | 244 |
| 45 | TGATCCTTTTACCTCCT | 2−13−2 | 306 | TGatccttttacctcCT | 267_1 | −22.2 | 308 | 305 |
| 46 | ACTGATCCTTTTACCTC | 2−12−3 | 307 | ACtgatccttttacCTC | 268_1 | −21.5 | 310 | 307 |
| 47 | CACTGATCCTTTTACCTC | 2−14−2 | 308 | CActgatccttttaccTC | 269_1 | −21.8 | 310 | 307 |
| 48 | ACACTGATCCTTTTACCT | 2−14−2 | 309 | ACactgatccttttacCT | 270_1 | −21.3 | 311 | 308 |
| 49 | CACTGATCCTTTTACCT | 2−13−2 | 310 | CActgatccttttacCT | 271_1 | −20.9 | 311 | 308 |
| 50 | AACACTGATCCTTTTACCT | 2−15−2 | 311 | AAcactgatccttttacCT | 272_1 | −21 | 311 | 308 |
| 51 | CACTGATCCTTTTACC | 3−11−2 | 312 | CACtgatccttttaCC | 273_1 | −21.2 | 312 | 309 |
| 52 | AACACTGATCCTTTTACC | 2−14−2 | 313 | AAcactgatccttttaCC | 274_1 | −19.9 | 312 | 309 |
| 53 | ACACTGATCCTTTTACC | 2−13−2 | 314 | ACactgatccttttaCC | 275_1 | −20.2 | 312 | 309 |
| 54 | AGCACAAAGTCATCTCC | 2−13−2 | 315 | AGcacaaagtcatctCC | 276_1 | −20.8 | 388 | 385 |
| 55 | TCACATAACAAGCTCC | 4−10−2 | 316 | TCACataacaagctCC | 277_1 | −20.4 | 495 | 492 |
| 56 | CCTATGTCACATAACAA | 4−9−4 | 317 | CCTAtgtcacataACAA | 278_1 | −22.3 | 500 | 497 |
| 57 | GCCTATGTCACATAACA | 2−13−2 | 318 | GCctatgtcacataaCA | 279_1 | −20.5 | 501 | 498 |
| 58 | AGCCTATGTCACATAAC | 2−11−4 | 319 | AGcctatgtcacaTAAC | 280_1 | −20.4 | 502 | 499 |
| 59 | AGCAAAACTAAACAATAAAAC | 4−13−4 | 320 | AGCAaaactaaacaataAA | 281_1 | −17.7 | 603 | 600 |
| | | | | AC | | | | |
| 60 | AGCAAAACTAAACAATAAA | 4−11−4 | 321 | AGCAaaactaaacaaTAA | 282_1 | −17.2 | 605 | 602 |
| | | | | A | | | | |
| 61 | AATCTAGGTTACACCC | 2−11−3 | 322 | AAtctaggttacaCCC | 283_1 | −20.5 | 641 | 638 |
| 62 | CCGATAACGAACTAA | 4−7−4 | 323 | CCGAtaacgaaCTAA | 284_1 | −19.6 | 1281 | 1277 |
| 63 | ACCCGATAACGAACT | 3−8−4 | 324 | ACCcgataacgAACT | 285_1 | −20.2 | 1283 | 1279 |
| 64 | CGCATCTTTTGATCC | 3−10−2 | 325 | CGCatcttttgatCC | 286_1 | −21 | 1307 | 1303 |
| 65 | AAGTAAATATCTCCTTCTT | 3−12−4 | 326 | AAGtaaatatctcctTCTT | 287_1 | −21 | 1459 | 1455 |
| 66 | AAGTAAATATCTCCTTCT | 3−11−4 | 327 | AAGtaaatatctccTTCT | 288_1 | −20.2 | 1460 | 1456 |
| 67 | AAGTAAATATCTCCTTC | 3−10−4 | 328 | AAGtaaatatctcCTTC | 289_1 | −18.3 | 1461 | 1457 |
| 68 | GAAGTAAATATCTCCTTC | 4−11−3 | 329 | GAAGtaaatatctccTTC | 290_1 | −20 | 1461 | 1457 |
| 69 | ACCCATAGACAACTTTA | 3−12−2 | 330 | ACCcatagacaacttTA | 291_1 | −20 | 1726 | 1722 |
| 70 | CACCCATAGACAACTTTA | 2−14−2 | 331 | CAcccatagacaacttTA | 292_1 | −19.9 | 1726 | 1722 |
| 71 | CACCCATAGACAACTTT | 4−11−2 | 332 | CACCcatagacaactTT | 293_1 | −21.9 | 1727 | 1723 |
| 72 | CCACCCATAGACAACTT | 2−13−2 | 333 | CCacccatagacaacTT | 294_1 | −21.3 | 1728 | 1724 |
| 73 | CACCCATAGACAACTT | 3−11−2 | 334 | CACccatagacaacTT | 295_1 | −18.8 | 1728 | 1724 |
| 74 | CCACCCATAGACAACT | 2−12−2 | 335 | CCacccatagacaaCT | 296_1 | −21.1 | 1729 | 1725 |
| 75 | ACCCACCCATAGACAAC | 2−12−3 | 336 | ACccacccatagacAAC | 297_1 | −21.5 | 1730 | 1726 |
| 76 | AGCTACCCACCGACA | 2−11−2 | 337 | AGctacccaccgaCA | 298_1 | −22.3 | 1776 | 1772 |
| 77 | CATACTTCTTCACTTCAAA | 2−13−4 | 338 | CAtacttcttcacttCAAA | 299_1 | −20 | 1881 | 1877 |
| 78 | ACCATACTTCTTCACTTCAAA | 2−15−4 | 339 | ACcatacttcttcacttCAAA | 300_1 | −23.6 | 1881 | 1877 |
| 79 | ATACTTCTTCACTTCAAA | 3−11−2 | 340 | ATActtcttcacttCAAA | 301_1 | −19.6 | 1881 | 1877 |
| 80 | CACCATACTTCTTCACTTCAA | 2−17−2 | 341 | CAccatacttcttcacttcAA | 302_1 | −22.8 | 1882 | 1878 |
| 81 | CATACTTCTTCACTTCAA | 4−11−3 | 342 | CATActtcttcacttCAA | 303_1 | −22.1 | 1882 | 1878 |
| 82 | ACCATACTTCTTCACTTCAA | 2−16−2 | 343 | ACcatacttcttcacttcAA | 304_1 | −20.7 | 1882 | 1878 |
| 83 | CCATACTTCTTCACTTCAA | 2−15−2 | 344 | CCatacttcttcacttcAA | 305_1 | −20.9 | 1882 | 1878 |
| 34 | ACCATACTTCTTCACTTCA | 2−15−2 | 345 | ACcatacttcttcacttCA | 306_1 | −22.1 | 1883 | 1879 |
| 85 | CATACTTCTTCACTTCA | 3−12−2 | 346 | CATacttcttcacttCA | 307_1 | −20.2 | 1883 | 1879 |
| 86 | CCATACTTCTTCACTTCA | 2−14−2 | 347 | CCatacttcttcacttCA | 308_1 | −22.2 | 1883 | 1879 |
| 87 | ACCATACTTCTTCACTTC | 3−13−2 | 348 | ACCatacttcttcactTC | 309_1 | −21.9 | 1884 | 1880 |
| 88 | CACCATACTTCTTCACTTC | 2−15−2 | 349 | CAccatacttcttcactTC | 310_1 | −21.9 | 1884 | 1880 |
| 89 | ACCATACTTCTTCACTT | 4−11−2 | 350 | ACCAtacttcttcacTT | 311_1 | −21.9 | 1885 | 1881 |
| 90 | TCACCATACTTCTTCACTT | 3−14−2 | 351 | TCAccatacttcttcacTT | 312_1 | −22.9 | 1885 | 1881 |
| 91 | CACCATACTTCTTCACTT | 2−14−2 | 352 | CAccatacttcttcacTT | 313_1 | −20.4 | 1885 | 1881 |
| 92 | CACCATACTTCTTCACT | 2−13−2 | 353 | CAccatacttcttcaCT | 314_1 | −20.2 | 1886 | 1882 |
| 93 | TCACCATACTTCTTCACT | 3−13−2 | 354 | TCAccatacttcttcaCT | 315_1 | −22.7 | 1886 | 1882 |
| 94 | CACTCACCATACTTCTTCAC | 2−16−2 | 355 | CActcaccatacttcttcAC | 316_1 | −23.1 | 1887 | 1883 |
| 95 | TCACCATACTTCTTCAC | 4−11−2 | 356 | TCACcatacttcttcAC | 317_1 | −21 | 1887 | 1883 |
| 96 | ACTCACCATACTTCTTCA | 2−13−3 | 357 | ACtcaccatacttctTCA | 318_1 | −22.3 | 1888 | 1884 |
| 97 | CACTCACCATACTTCTTCA | 2−15−2 | 358 | CActcaccatacttcttCA | 319_1 | −23.2 | 1888 | 1884 |
| 98 | CTCACCATACTTCTTCA | 3−12−2 | 359 | CTCaccatacttcttCA | 320_1 | −21.8 | 1888 | 1884 |
| 99 | CACTCACCATACTTCTTC | 2−14−2 | 360 | CActcaccatacttctTC | 321_1 | −20.9 | 1889 | 1885 |
| 100 | CACTCACCATACTTCTT | 2−13−2 | 361 | CActcaccatacttcTT | 322_1 | −19.4 | 1890 | 1886 |
| 101 | GCACTCACCATACTTCT | 2−13−2 | 362 | GCactcaccatacttCT | 323_1 | −22.7 | 1891 | 1887 |
| 102 | TCAGCACTCACCATACTTC | 2−15−2 | 363 | TCagcactcaccatactTC | 324_1 | −22.8 | 1892 | 1888 |
| 103 | GCACTCACCATACTTC | 2−12−2 | 364 | GCactcaccatactTC | 325_1 | −20.2 | 1892 | 1888 |
| 104 | AGCACTCACCATACTTC | 2−13−2 | 365 | AGcactcaccatactTC | 326_1 | −20.4 | 1892 | 1888 |
| 105 | TCAGCACTCACCATACTT | 2−14−2 | 366 | TCagcactcaccatacTT | 327_1 | −21.4 | 1893 | 1889 |
| 106 | CAGCACTCACCATACTT | 2−13−2 | 367 | CAgcactcaccatacTT | 328_1 | −20.8 | 1893 | 1889 |
| 107 | AGCACTCACCATACTT | 2−12−2 | 368 | AGcactcaccatacTT | 329_1 | −19 | 1893 | 1889 |
| 108 | ACTTCAGCACTCACCAT | 2−12−3 | 369 | ACttcagcactcacCAT | 330_1 | −22.8 | 1897 | 1893 |
| 109 | GAGTAATCTTCACCTC | 2−11−3 | 370 | GAgtaatcttcacCTC | 331_1 | −19.6 | 2014 | 2010 |
| 110 | TAATTGGATTTCATCAC | 4−9−4 | 371 | TAATtggatttcaTCAC | 332_1 | −19.3 | 2066 | 2062 |
| 111 | GACGCTACCCTACCTT | 2−12−2 | 372 | GAcgctaccctaccTT | 333_1 | −22.5 | 2733 | 2711 |
| 112 | TGACGCTACCCTACCT | 2−12−2 | 373 | TGacgctaccctacCT | 334_1 | −22.9 | 2734 | 2712 |
| 113 | GACGCTACCCTACCT | 2−11−2 | 374 | GAcgctaccctacCT | 335_1 | −22.2 | 2734 | 2712 |
| 114 | TTGACGCTACCCTACC | 2−12−2 | 375 | TTgacgctaccctaCC | 336_1 | −22.5 | 2735 | 2713 |
| 115 | TTGACGCTACCCTAC | 2−9−4 | 376 | TTgacgctaccCTAC | 337_1 | −21.5 | 2736 | 2714 |
| 116 | CTTGACGCTACCCTAC | 2−12−2 | 377 | CTtgacgctaccctAC | 338_1 | −20.1 | 2736 | 2714 |
| 117 | CTTGACGCTACCCTA | 2−11−2 | 378 | CTtgacgctacccTA | 339_1 | −20.1 | 2737 | 2715 |
| 118 | CCTTCCACCAACTAAG | 2−12−2 | 379 | CCttccaccaactaAG | 340_1 | −20.6 | 2806 | 2784 |
| 119 | AATATTGATTTTATCCA | 4−9−4 | 380 | AATAttgattttaTCCA | 341_1 | −19.9 | 2866 | 2844 |
| 120 | CAATATTGATTTTATCC | 3−10−4 | 381 | CAAtattgattttATCC | 342_1 | −18 | 2867 | 2845 |
| 121 | CCAATATTGATTTTATCC | 2−13−3 | 382 | CCaatattgattttaTCC | 343_1 | −20.3 | 2867 | 2845 |
| 122 | TCTCTGACCCCAATATT | 2−13−2 | 383 | TCtctgaccccaataTT | 344_1 | −20.4 | 2877 | 2855 |
| 123 | CATTTGCATTTTTAAACT | 4−10−4 | 384 | CATTtgcatttttaAACT | 345_1 | −19.6 | 3005 | 2981 |
| 124 | CCATTTGCATTTTTAAAC | 4−12−2 | 385 | CCATttgcatttttaaAC | 346_1 | −19.9 | 3006 | 2982 |
| 125 | AACAACTCATGCACCC | 3−11−2 | 386 | AACaactcatgcacCC | 347_1 | −20 | 3723 | 3659 |
| 126 | CCCAGGAAACAACTCAT | 2−12−3 | 387 | CCcaggaaacaactCAT | 348_1 | −21.6 | 3729 | 3665 |
| 127 | CACCCAGGAAACAACTC | 3−12−2 | 388 | CACccaggaaacaacTC | 349_1 | −20.5 | 3731 | 3667 |
| 128 | CAAGTCCCACATACCAT | 2−13−2 | 389 | CAagtcccacataccAT | 350_1 | −20.9 | 4004 | 3945 |
| 129 | TATACCCCAAGTCCCAC | 2−13−2 | 390 | TAtaccccaagtcccAC | 351_1 | −22.9 | 4011 | 3952 |
| 130 | ACTCCTATACCCCAAG | 2−12−2 | 391 | ACtcctataccccaAG | 352_1 | −20.9 | 4017 | 3958 |
| 131 | CTAAACCAGTCATCCT | 3−11−2 | 392 | CTAaaccagtcatcCT | 353_1 | −20.4 | 4502 | 4453 |
| 132 | GCTAAACCAGTCATCC | 2−12−2 | 393 | GCtaaaccagtcatCC | 354_1 | −21.5 | 4503 | 4454 |
| 133 | CTGTTGATTACTTCAAA | 4−9−4 | 394 | CTGTtgattacttCAAA | 355_1 | −19.9 | 5514 | 6127 |
| 134 | TAGTCGATCACCATCA | 2−11−3 | 395 | AgtcgatcaccaTCA | 356_1 | −20.4 | 5606 | 6219 |
| 135 | TTAGTCGATCACCATC | 2−10−4 | 396 | TTagtcgatcacCATC | 357_1 | −20.2 | 5607 | 6220 |
| 136 | TAGTCGATCACCATC | 2−10−3 | 397 | TAgtcgatcaccATC | 358_1 | −18 | 5607 | 6220 |
| 137 | TTAGTCGATCACCAT | 3−8−4 | 398 | TTAgtcgatcaCCAT | 359_1 | −21.7 | 5608 | 6221 |
| 138 | CTACGTTCTTACCCTCT | 2−13−2 | 399 | CTacgttcttaccctCT | 360_1 | −22.9 | 5633 | 6246 |
| 139 | TACGTTCTTACCCTCT | 2−11−3 | 400 | TAcgttcttacccTCT | 361_1 | −21.9 | 5633 | 6246 |
| 140 | CACTACGTTCTTACCCTC | 2−14−2 | 401 | CActacgttcttacccTC | 362_1 | −23 | 5634 | 6247 |
| 141 | CTACGTTCTTACCCTC | 2−12−2 | 402 | CTacgttcttacccTC | 363_1 | −20.7 | 5634 | 6247 |
| 142 | ACTACGTTCTTACCCTC | 2−12−3 | 403 | ACtacgttcttaccCTC | 364_1 | −22.6 | 5634 | 6247 |
| 143 | CACTACGTTCTTACCCT | 2−13−2 | 404 | CActacgttcttaccCT | 365_1 | −22.1 | 5635 | 6248 |
| 144 | ACTACGTTCTTACCCT | 2−12−2 | 405 | ACtacgttcttaccCT | 366_1 | −20.1 | 5635 | 6248 |
| 145 | ACTACGTTCTTACCC | 3−10−2 | 406 | ACTacgttcttacCC | 367_1 | −20.6 | 5636 | 6249 |
| 146 | TCACTACGTTCTTACCC | 2−13−2 | 407 | TCactacgttcttacCC | 368_1 | −21.8 | 5636 | 6249 |
| 147 | CACTACGTTCTTACCC | 3−11−2 | 408 | CACtacgttcttacCC | 369_1 | −22.2 | 5636 | 6249 |
| 148 | TCACTACGTTCTTACC | 2−11−3 | 409 | TCactacgttcttACC | 370_1 | −19.6 | 5637 | 6250 |
| 149 | CACTACGTTCTTACC | 2−11−2 | 410 | CActacgttcttaCC | 371_1 | −18 | 5637 | 6250 |
| 150 | CTCACTACGTTCTTACC | 2−13−2 | 411 | CTcactacgttcttaCC | 372_1 | −21.2 | 5637 | 6250 |
| 151 | CACTCACTACGTTCTTAC | 3−13−2 | 412 | CACtcactacgttcttAC | 373_1 | −20.7 | 5638 | 6251 |
| 152 | ACTCACTACGTTCTTAC | 4−10−3 | 413 | ACTCactacgttctTAC | 374_1 | −21.7 | 5638 | 6251 |
| 153 | TCACTACGTTCTTAC | 3−9−3 | 414 | TCActacgttctTAC | 375_1 | −17.7 | 5638 | 6251 |
| 154 | CTCACTACGTTCTTAC | 3−10−3 | 415 | CTCactacgttctTAC | 376_1 | −19.6 | 5638 | 6251 |
| 155 | CCACTCACTACGTTCTTA | 2−14−2 | 416 | CCactcactacgttctTA | 377_1 | −22.6 | 5639 | 6252 |
| 156 | CTCACTACGTTCTTA | 4−7−4 | 417 | CTCActacgttCTTA | 378_1 | −21.8 | 5639 | 6252 |
| 157 | ACTCACTACGTTCTTA | 2−11−3 | 418 | ACtcactacgttcTTA | 379_1 | −18.1 | 5639 | 6252 |
| 158 | CACTCACTACGTTCTT | 2−10−4 | 419 | CActcactacgtTCTT | 380_1 | −20.4 | 5640 | 6253 |
| 159 | CCACTCACTACGTTCT | 2−11−3 | 420 | CCactcactacgtTCT | 381_1 | −22.2 | 5641 | 6254 |
| 160 | CCCACTCACTACGTTC | 2−12−2 | 421 | CCcactcactacgtTC | 382_1 | −21.8 | 5642 | 6255 |
| 161 | CCACTCACTACGTTC | 4−9−2 | 422 | CCACtcactacgtTC | 383_1 | −21.7 | 5642 | 6255 |
| 162 | CCCACTCACTACGTT | 2−11−2 | 423 | CCcactcactacgTT | 384_1 | −20.4 | 5643 | 6256 |
| 163 | AGTTAACATTTCTCTTTT | 3−11−4 | 424 | AGTtaacatttctcTTTT | 385_1 | −19.8 | 5883 | 6947 |
| 164 | AAGTTAACATTTCTCTTT | 4−10−4 | 425 | AAGTtaacatttctCTTT | 386_1 | −20.4 | 5884 | 6948 |
| 165 | CAACATACACTTCTCACT | 2−14−2 | 426 | CAacatacacttctcaCT | 387_1 | −19.2 | 5915 | 6979 |
| 166 | CACAACATACACTTCTCACT | 2−16−2 | 427 | CAcaacatacacttctcaCT | 388_1 | −21.9 | 5915 | 6979 |
| 167 | AACATACACTTCTCACT | 3−10−4 | 428 | AACatacacttctCACT | 389_1 | −20.2 | 5915 | 6979 |
| 168 | ACAACATACACTTCTCACT | 2−15−2 | 429 | ACaacatacacttctcaCT | 390_1 | −19.7 | 5915 | 6979 |
| 169 | CCACAACATACACTTCTCAC | 2−16−2 | 430 | CCacaacatacacttctcAC | 391_1 | −22.6 | 5916 | 6980 |
| 170 | CACAACATACACTTCTCAC | 2−15−2 | 431 | CAcaacatacacttctcAC | 392_1 | −19 | 5916 | 6980 |
| 171 | CACAACATACACTTCTCA | 3−13−2 | 432 | CACaacatacacttctCA | 393_1 | −20.1 | 5917 | 6981 |
| 172 | CCACAACATACACTTCTCA | 2−15−2 | 433 | CCacaacatacacttctCA | 394_1 | −22.7 | 5917 | 6981 |
| 173 | CACAACATACACTTCTC | 4−9−4 | 434 | CACAacatacactTCTC | 395_1 | −22.2 | 5918 | 6982 |
| 174 | CCACAACATACACTTCTC | 2−14−2 | 435 | CCacaacatacacttcTC | 396_1 | −20.6 | 5918 | 6982 |
| 175 | CCCACAACATACACTTCT | 2−14−2 | 436 | CCcacaacatacacttCT | 397_1 | −22.7 | 5919 | 6983 |
| 176 | ACCCACAACATACACTTCT | 2−15−2 | 437 | ACccacaacatacacttCT | 398_1 | −22.5 | 5919 | 6983 |
| 177 | CACCCACAACATACACTTC | 2−15−2 | 438 | CAcccacaacatacactTC | 399_1 | −22.1 | 5920 | 6984 |
| 178 | ACCCACAACATACACTTC | 2−14−2 | 439 | ACccacaacatacactTC | 400_1 | −20 | 5920 | 6984 |
| 179 | CCCACAACATACACTTC | 2−13−2 | 440 | CCcacaacatacactTC | 401_1 | −20.2 | 5920 | 6984 |
| 180 | CACCCACAACATACACTT | 3−13−2 | 441 | CACccacaacatacacTT | 402_1 | −21.8 | 5921 | 6985 |
| 181 | ACCCACAACATACACTT | 3−11−3 | 442 | ACCcacaacatacaCTT | 403_1 | −22.5 | 5921 | 6985 |
| 182 | CACCCACAACATACACT | 2−13−2 | 443 | CAcccacaacatacaCT | 404_1 | −20.5 | 5922 | 6986 |
| 183 | GGCACCCACAACATACA | 2−13−2 | 444 | GGcacccacaacataCA | 405_1 | −22.8 | 5924 | 6988 |
| 184 | CTCCATACCACAACTG | 3−10−3 | 445 | CTCcataccacaaCTG | 406_1 | −22.1 | 6012 | 7076 |
| 185 | CCTCCATACCACAACT | 2−12−2 | 446 | CCtccataccacaaCT | 407_1 | −22.2 | 6013 | 7077 |
| 186 | ACCCTCCATACCACAAC | 2−13−2 | 447 | ACcctccataccacaAC | 408_1 | −22 | 6014 | 7078 |
| 187 | CCCTCCATACCACAAC | 2−12−2 | 448 | CCctccataccacaAC | 409_1 | −22.2 | 6014 | 7078 |
| 188 | CACCCTCCATACCACAA | 2−13−2 | 449 | CAccctccataccacAA | 410_1 | −22.3 | 6015 | 7079 |
| 189 | CAAACCTAGAACCACCA | 2−13−2 | 450 | CAaacctagaaccacCA | 411_1 | −19.8 | 6105 | 7170 |
| 190 | CAAACCTAGAACCACC | 4−10−2 | 451 | CAAAcctagaaccaCC | 412_1 | −20 | 6106 | 7171 |
| 191 | TCAAACCTAGAACCACC | 3−11−3 | 452 | TCAaacctagaaccACC | 413_1 | −21.9 | 6106 | 7171 |
| 192 | ATCAAACCTAGAACCAC | 3−10−4 | 453 | ATCaaacctagaaCCAC | 414_1 | −21.7 | 6107 | 7172 |
| 193 | CATCAAACCTAGAACCAC | 2−13−3 | 454 | CAtcaaacctagaacCAC | 415_1 | −20 | 6107 | 7172 |
| 194 | TCAAACCTAGAACCAC | 3−10−3 | 455 | TCAaacctagaacCAC | 416_1 | −18.7 | 6107 | 7172 |
| 195 | CATCAAACCTAGAACC | 3−9−4 | 456 | CATcaaacctagAACC | 417_1 | −19.9 | 6109 | 7174 |
| 196 | ICCATCAAACCTAGAAC | 3−10−3 | 457 | CCAtcaaacctagAAC | 418_1 | −18.6 | 6110 | 7175 |
| 197 | AGCATCCATCAAACCTA | 2−12−3 | 458 | AGcatccatcaaacCTA | 419_1 | −21.9 | 6114 | 7179 |
| 198 | GCATCCATCAAACCTA | 2−11−3 | 459 | GCatccatcaaacCTA | 420_1 | −21.6 | 6114 | 7179 |
| 199 | CAGCATCCATCAAACCT | 2−13−2 | 460 | CAgcatccatcaaacCT | 421_1 | −21.5 | 6115 | 7180 |
| 200 | TCAGCATCCATCAAACC | 2−13−2 | 461 | TCagcatccatcaaaCC | 422_1 | −20.9 | 6116 | 7181 |
| 201 | CAGCAAACTTGCAACA | 3−9−4 | 462 | CAGcaaacttgcAACA | 423_1 | −19.9 | 6308 | 7373 |
| 202 | TCTACAGGTTCCACTC | 2−12−2 | 463 | TCtacaggttccacTC | 424_1 | −19.7 | 6542 | 7616 |
| 203 | AACACTCGAACACGA | 4−7−4 | 464 | AACActcgaacACGA | 425_1 | −18.8 | 6956 | 8030 |
| 204 | CCCCTACGGTCTCAC | 2−11−2 | 465 | CCcctacggtctcAC | 426_1 | −22.6 | 7074 | 8148 |
| 205 | TCCCCTACGGTCTCAC | 2−12−2 | 466 | TCccctacggtctcAC | 427_1 | −22.9 | 7074 | 8148 |
| 206 | TCACATGGACACTCACC | 2−13−2 | 467 | TCacatggacactcaCC | 428_1 | −21.4 | 7201 | 8275 |
| 207 | ATTGTAAGTTTATCCA | 4−9−3 | 468 | ATTGtaagtttatCCA | 429_1 | −20 | 7506 | 8580 |
| 208 | CAATTGAGTCACAATA | 4−8−4 | 469 | CAATtgagtcacAATA | 430_1 | −17 | 7526 | 8600 |
| 209 | CTCAATATAAGTAAACAA | 4−10−4 | 470 | CTCAatataagtaaACAA | 431_1 | −16.9 | 7539 | 8627 |
| 210 | GCCTCTGTCTCAATATA | 2−13−2 | 471 | GCctctgtctcaataTA | 432_1 | −21.2 | 7548 | 8636 |
| 211 | AGACTAGTAATTCCAAA | 4−9−4 | 472 | AGACtagtaattcCAAA | 433_1 | −19.7 | 7648 | 8711 |
| 212 | TTCTCTACCATGTACAA | 3−11−3 | 473 | TTCtctaccatgtaCAA | 434_1 | −19.9 | 7758 | 8821 |
| 213 | CTTAACCCAGAACCTTT | 2−12−3 | 474 | CTtaacccagaaccTTT | 435_1 | −20.2 | 8436 | 9514 |
| 214 | CCTTAACCCAGAACCTT | 2−13−2 | 475 | CCttaacccagaaccTT | 436_1 | −22.3 | 8437 | 9515 |
| 215 | TTCCCTTAACCCAGAAC | 3−12−2 | 476 | TTCccttaacccagaAC | 437_1 | −21.4 | 8440 | 9518 |
| 216 | CAAGGTTCCTACTCTC | 3−11−2 | 477 | CAAggttcctactcTC | 438_1 | −20 | 8564 | 9642 |
| 217 | CCAAACTCAGAATCTTCA | 2−14−2 | 478 | CCaaactcagaatcttCA | 439_1 | −19.7 | 8794 | 9872 |
| 218 | TGCTGACATCCTACAC | 2−11−3 | 479 | TGctgacatcctaCAC | 440_1 | −20.5 | 8955 | 10033 |
| 219 | CTGCTGACATCCTACAC | 2−13−2 | 480 | CTgctgacatcctacAC | 441_1 | −20.6 | 8955 | 10033 |
| 220 | CACGGCTTCCTTACCAC | 2−13−2 | 481 | CAcggcttccttaccAC | 442_1 | −22.9 | 9141 | 10221 |
| 221 | CACGGCTTCCTTACCA | 2−12−2 | 482 | CAcggcttccttacCA | 443_1 | −22.9 | 9142 | 10222 |
| 222 | CAACACAACTTTGTCCT | 3−12−2 | 483 | CAAcacaactttgtcCT | 444_1 | −19.5 | 9631 | 10711 |
| 223 | AATACCCCAGCAACCC | 2−12−2 | 484 | AAtaccccagcaacCC | 445_1 | −22.4 | 10098 | 11182 |
| 224 | CAATACCCCAGCAACC | 2−12−2 | 485 | CAataccccagcaaCC | 446_1 | −22.3 | 10099 | 11183 |
| 225 | CCAATACCCCAGCAAC | 2−12−2 | 486 | CCaataccccagcaAC | 447_1 | −21.7 | 10100 | 11184 |
| 226 | CCCAATACCCCAGCAA | 2−12−2 | 487 | CCcaataccccagcAA | 448_1 | −22.9 | 10101 | 11185 |
| 227 | AACAATGAAATATATTCAT | 4−11−4 | 488 | AACAatgaaatatatTCAT | 449_1 | −16.8 | 10342 | 11425 |
| 228 | ACAATGAAATATATTCAT | 4−10−4 | 489 | ACAAtgaaatatatTCAT | 450_1 | −16.6 | 10342 | 11425 |
| 229 | GATATTACAATTCATAACTA | 3−13−4 | 490 | GATattacaattcataACTA | 451_1 | −20.2 | 10382 | 11465 |
| 230 | CAGATATTACAATTCATAACT | 3−16−2 | 491 | CAGatattacaattcataaCT | 452_1 | −19.6 | 10383 | 11466 |
| 231 | GATATTACAATTCATAACT | 3−13−3 | 492 | GATattacaattcataACT | 453_1 | −18 | 10383 | 11466 |
| 232 | AGATATTACAATTCATAACT | 4−12−4 | 493 | AGATattacaattcatAACT | 454_1 | −20.9 | 10383 | 11466 |
| 233 | GTCAGATATTACAATTCA | 3−11−4 | 494 | GTCagatattacaaTTCA | 455_1 | −20 | 10388 | 11471 |
| 234 | AATCTCCAGAACAACTA | 4−10−3 | 495 | AATCtccagaacaaCTA | 456_1 | −20 | 10462 | 11545 |
| 235 | CAATCTCCAGAACAACT | 2−11−4 | 496 | CAatctccagaacAACT | 457_1 | −18.8 | 10463 | 11546 |
| 236 | TCAATCTCCAGAACAAC | 4−9−4 | 497 | TCAAtctccagaaCAAC | 458_1 | −20.4 | 10464 | 11547 |
| 237 | TACAGTGTCAATCTCCA | 2−13−2 | 498 | TAcagtgtcaatctcCA | 459_1 | −19.8 | 10471 | 11554 |
| 238 | CTACAGTGTCAATCTC | 3−10−3 | 499 | CTAcagtgtcaatCTC | 460_1 | −20 | 10473 | 11556 |
| 239 | TAGTAGCCCCACACAC | 2−12−2 | 500 | TAgtagccccacacAC | 461_1 | −21.5 | 10517 | 11588 |
| 240 | CCCCTGTACACTTTAC | 2−12−2 | 501 | CCcctgtacactttAC | 462_1 | −20.9 | 10763 | 11879 |
| 241 | CTACCAAGACATCTAT | 2−10−4 | 502 | CTaccaagacatCTAT | 463_1 | −19.7 | 11188 | 12300 |
| 242 | GCTACCAAGACATCTA | 2−12−2 | 503 | GCtaccaagacatcTA | 464_1 | −19.3 | 11189 | 12301 |
| 243 | TGACCTTCACTTCTATC | 4−11−2 | 504 | TGACcttcacttctaTC | 465_1 | −21.9 | 11658 | 12770 |
| 244 | CACTCACCAGATACACACA | 2−15−2 | 505 | CActcaccagatacacaCA | 466_1 | −22.7 | 11951 | 13063 |
| 245 | ACTCACCAGATACACACA | 2−14−2 | 506 | ACtcaccagatacacaCA | 467_1 | −20.8 | 11951 | 13063 |
| 246 | TCTCTTTACTCACCGA | 2−11−3 | 507 | TCtctttactcacCGA | 468_1 | −21 | 12349 | 13461 |
| 247 | GAAACTTCTCTTTACTCACC | 2−16−2 | 508 | GAaacttctctttactcaCC | 469_1 | −22.8 | 12351 | 13463 |
| 248 | ACTTCTCTTTACTCACC | 3−12−2 | 509 | ACTtctctttactcaCC | 470_1 | −21.8 | 12351 | 13463 |
| 249 | AAACTTCTCTTTACTCACC | 2−15−2 | 510 | AAacttctctttactcaCC | 471_1 | −20.1 | 12351 | 13463 |
| 250 | AACTTCTCTTTACTCACC | 2−14−2 | 511 | AActtctctttactcaCC | 472_1 | −20 | 12351 | 13463 |
| 251 | GAAACTTCTCTTTACTCAC | 3−12−4 | 512 | GAAacttctctttacTCAC | 473_1 | −22 | 12352 | 13464 |
| 252 | AACTTCTCTTTACTCAC | 4−9−4 | 513 | AACTtctctttacTCAC | 474_1 | −21.2 | 12352 | 13464 |
| 253 | AAACTTCTCTTTACTCAC | 3−11−4 | 514 | AAActtctctttacTCAC | 475_1 | −19.4 | 12352 | 13464 |
| 254 | TGAAACTTCTCTTTACTCAC | 2−16−2 | 515 | TGaaacttctctttactcAC | 476_1 | −18.9 | 12352 | 13464 |
| 255 | AAACTTCTCTTTACTCA | 4−9−4 | 516 | AAACttctctttaCTCA | 477_1 | −20.4 | 12353 | 13465 |
| 256 | GAAACTTCTCTTTACTCA | 2−14−2 | 517 | GAaacttctctttactCA | 478_1 | −18.2 | 12353 | 13465 |
| 257 | TGAAACTTCTCTTTACTCA | 3−13−3 | 518 | TGAaacttctctttacTCA | 479_1 | −21.9 | 12353 | 13465 |
| 258 | GAAACTTCTCTTTACTC | 4−10−3 | 519 | GAAActtctctttaCTC | 480_1 | −18.9 | 12354 | 13466 |
| 259 | TGAAACTTCTCTTTACTC | 3−12−3 | 520 | TGAaacttctctttaCTC | 481_1 | −20.3 | 12354 | 13466 |
| 260 | TGAAACTTCTCTTTACT | 3−12−2 | 521 | TGAaacttctctttaCT | 482_1 | −17.8 | 12355 | 13467 |
| 261 | GTGAAACTTCTCTTTACT | 3−13−2 | 522 | GTGaaacttctctttaCT | 483_1 | −19.8 | 12355 | 13467 |
| 262 | TACAGACTCAAAAACCCA | 3−12−3 | 523 | TACagactcaaaaacCCA | 484_1 | −21.8 | 12385 | 13497 |
| 263 | ACAGACTCAAAAACCCA | 3−12−2 | 524 | ACAgactcaaaaaccCA | 485_1 | −19.2 | 12385 | 13497 |
| 264 | ATACAGACTCAAAAACCCA | 3−14−2 | 525 | ATAcagactcaaaaaccCA | 486_1 | −20.5 | 12385 | 13497 |
| 265 | CATACAGACTCAAAAACCC | 2−14−3 | 526 | CAtacagactcaaaaaCCC | 487_1 | −22.2 | 12386 | 13498 |
| 266 | ATACAGACTCAAAAACCC | 3−13−2 | 527 | ATAcagactcaaaaacCC | 488_1 | −19.4 | 12386 | 13498 |
| 267 | TACAGACTCAAAAACCC | 2−12−3 | 528 | TAcagactcaaaaaCCC | 489_1 | −19.8 | 12386 | 13498 |
| 268 | CATACAGACTCAAAAACC | 2−12−4 | 529 | CAtacagactcaaaAACC | 490_1 | −18.4 | 12387 | 13499 |
| 269 | ATACAGACTCAAAAACC | 4−9−4 | 530 | ATACagactcaaaAACC | 491_1 | −19.1 | 12387 | 13499 |
| 270 | TCATACAGACTCAAAAAC | 4−10−4 | 531 | TCATacagactcaaAAAC | 492_1 | −17.9 | 12388 | 13500 |
| 271 | ATCATACAGACTCAAAAAC | 4−11−4 | 532 | ATCAtacagactcaaAAAC | 493_1 | −18.3 | 12388 | 13500 |
| 272 | ACCCTTATCATACAGA | 3−11−2 | 533 | ACCcttatcatacaGA | 494_1 | −20.5 | 12397 | 13509 |
| 273 | TGACCCTTATCATACA | 2−12−2 | 534 | TGacccttatcataCA | 495_1 | −18.3 | 12399 | 13511 |
| 274 | ACTAGACTCTAAAATCT | 4−9−4 | 535 | ACTAgactctaaaATCT | 496_1 | −20.1 | 12725 | 13837 |
| 275 | CCACTAGACTCTAAAATCT | 2−15−2 | 536 | CCactagactctaaaatCT | 497_1 | −20.1 | 12725 | 13837 |
| 276 | CACTAGACTCTAAAATCT | 3−13−2 | 537 | CACtagactctaaaatCT | 498_1 | −17.9 | 12725 | 13837 |
| 277 | CACTAGACTCTAAAATC | 4−9−4 | 538 | CACTagactctaaAATC | 499_1 | −18.8 | 12726 | 13838 |
| 278 | CCACTAGACTCTAAAATC | 2−14−2 | 539 | CCactagactctaaaaTC | 500_1 | −17.7 | 12726 | 13838 |
| 279 | CCACTAGACTCTAAAAT | 4−9−4 | 540 | CCACtagactctaAAAT | 501_1 | −20.4 | 12727 | 13839 |
| 280 | CACTGGCATACATCTCC | 2−13−2 | 541 | CActggcatacatctCC | 502_1 | −22.5 | 13154 | 14283 |
| 281 | ACTGGCATACATCTCC | 2−12−2 | 542 | ACtggcatacatctCC | 503_1 | −20.6 | 13154 | 14283 |
| 282 | AGGCGAACCTCATCC | 2−11−2 | 543 | AGgcgaacctcatCC | 504_1 | −21.8 | 13316 | 14445 |
| 283 | ACTGACCATACTCCACT | 2−13−2 | 544 | ACtgaccatactccaCT | 505_1 | −21.4 | 13344 | 14473 |
| 284 | GACTGACCATACTCCAC | 2−13−2 | 545 | GActgaccatactccAC | 506_1 | −20.4 | 13345 | 14474 |
| 285 | GACTGACCATACTCCA | 3−11−2 | 546 | GACtgaccatactcCA | 507_1 | −21.7 | 13346 | 14475 |
| 286 | AACCTTAACCGTGAA | 4−7−4 | 547 | AACCttaaccgTGAA | 508_1 | −20.8 | 13491 | 14620 |
| 287 | CGAAGAACCTTAACC | 4−7−4 | 548 | CGAAgaaccttAACC | 509_1 | −19.6 | 13496 | 14625 |
| 288 | TCGAAGAACCTTAACC | 2−10−4 | 549 | TCgaagaaccttAACC | 510_1 | −18.1 | 13496 | 14625 |
| 289 | TCGAAGAACCTTAAC | 4−7−4 | 550 | TCGAagaacctTAAC | 511_1 | −17.6 | 13497 | 14626 |
| 290 | TTCTCGAAGAACCTTA | 3−9−4 | 551 | TTCtcgaagaacCTTA | 512_1 | −19.7 | 13499 | 14628 |
| 291 | AGATTTCCCATTTCCAA | 3−12−2 | 552 | AGAtttcccatttccAA | 513_1 | −20.8 | 13643 | 14772 |
| 292 | GAGATTTCCCATTTCCAA | 2−14−2 | 553 | GAgatttcccatttccAA | 514_1 | −21.1 | 13643 | 14772 |
| 293 | GAGATTTCCCATTTCCA | 2−13−2 | 554 | GAgatttcccatttcCA | 515_1 | −22.3 | 13644 | 14773 |
| 294 | ACTTGTTGCTCACTAT | 2−11−3 | 555 | ACttgttgctcacTAT | 516_1 | −19.2 | 13732 | 14861 |
| 295 | CCATCCCCATGATCAA | 3−11−2 | 556 | CCAtccccatgatcAA | 517_1 | −22.7 | 13946 | 15075 |
| 296 | CTTTTCTTTTATTTACCCT | 3−14−2 | 557 | CTTttcttttatttaccCT | 518_1 | −22.2 | 14340 | 15469 |
| 297 | TTTTCTTTTATTTACCCT | 3−13−2 | 558 | TTTtcttttatttaccCT | 519_1 | −19.5 | 14340 | 15469 |
| 298 | GCTTTTCTTTTATTTACCC | 2−15−2 | 559 | GCttttcttttatttacCC | 520_1 | −24 | 14341 | 15470 |
| 299 | CTTTTCTTTTATTTACCC | 2−14−2 | 560 | CTtttcttttatttacCC | 521_1 | −20.2 | 14341 | 15470 |
| 300 | AAGCTTTTCTTTTATTTACC | 2−16−2 | 561 | AAgcttttcttttatttaCC | 522_1 | −20.4 | 14342 | 15471 |
| 301 | GCTTTTCTTTTATTTACC | 2−14−2 | 562 | GCttttcttttatttaCC | 523_1 | −21.2 | 14342 | 15471 |
| 302 | AGCTTTTCTTTTATTTACC | 2−14−3 | 563 | AGcttttcttttatttACC | 524_1 | −21.7 | 14342 | 15471 |

In the table, capital letters are beta-D-oxy LNA nucleosides, lowercase letters are DNA nucleosides, all LNA C are 5-methyl cytosine, and all internucleoside linkages are phosphorothioate internucleoside linkages.

The relative mouse C4b and mouse C4a mRNA expression level in Table 8 is shown as percent of control (PBS-treated cells). The values in the columns designated with underlined C4b are based on detection of C4b transcripts only. The values in the columns designated with underlined C4a and C4b are based on detection of C4a and C4b transcripts.

TABLE 8

| | | | C4 |
|---|---|---|---|
| | | C4 | mRNA |
| | | mRNA | qPCR SP |
| | | qPCR SP | probe1 |
| | | probe1 | mouse |
| | | mouse | hepato- |
| | | hepato- | cytes |
| CMP | | cytes | C4a |
| ID | | C4b: | and C4b: |
| NO | Conc | AP015278 | AP015277 |
| 264_1 | 0.3 | 33.2 | 24.4 |
| 265_1 | 0.06 | 89.1 | 78.6 |
| 265_1 | 0.3 | 45.6 | 39.5 |
| 266_1 | 0.06 | 109 | 96 |
| 266_1 | 0.3 | 52.8 | 50.4 |
| 267_1 | 0.06 | 85 | 73 |
| 267_1 | 0.3 | 44.8 | 38.6 |
| 268_1 | 0.06 | 82 | 83.7 |
| 268_1 | 0.3 | 42.1 | 34.5 |
| 269_1 | 0.06 | 96.6 | 92.6 |
| 269_1 | 0.3 | 53.1 | 47.1 |
| 270_1 | 0.06 | 107 | 91.9 |
| 270_1 | 0.3 | 44.7 | 34.8 |
| 271_1 | 0.06 | 83.1 | 72.9 |
| 271_1 | 0.3 | 55.9 | 46.2 |
| 272_1 | 0.06 | 104 | 106 |
| 272_1 | 0.3 | 63 | 54.7 |
| 273_1 | 0.06 | 90 | 71.7 |
| 273_1 | 0.3 | 43.7 | 35.9 |
| 274_1 | 0.06 | 68.1 | 52.3 |
| 274_1 | 0.3 | 44.1 | 37.1 |
| 275_1 | 0.06 | 118 | 107 |
| 275_1 | 0.3 | 48.3 | 36 |
| 276_1 | 0.06 | 104 | 102 |
| 276_1 | 0.3 | 50 | 44.8 |
| 277_1 | 0.06 | 73.7 | 59.2 |
| 277_1 | 0.3 | 40.9 | 33.6 |
| 278_1 | 0.06 | 104 | 92.4 |
| 278_1 | 0.3 | 33.7 | 29.3 |
| 279_1 | 0.06 | 78.8 | 75.4 |
| 279_1 | 0.3 | 41.3 | 34.8 |
| 280_1 | 0.06 | 48.6 | 39.3 |
| 280_1 | 0.3 | 47.5 | 45.8 |
| 281_1 | 0.06 | 73.2 | 70.7 |
| 281_1 | 0.3 | 36.4 | 31.7 |
| 282_1 | 0.06 | 59.4 | 42.4 |
| 282_1 | 0.3 | 60.5 | 52.8 |
| 283_1 | 0.06 | 164 | 183 |
| 283_1 | 0.3 | 75.9 | 64.3 |
| 284_1 | 0.06 | 118 | 120 |
| 284_1 | 0.3 | 81.4 | 69.3 |
| 285_1 | 0.06 | 63 | 49.6 |
| 285_1 | 0.3 | 72.4 | 65 |
| 286_1 | 0.06 | 40.9 | 35.1 |
| 286_1 | 0.3 | 20.9 | 17.3 |
| 287_1 | 0.06 | 80 | 63.4 |
| 287_1 | 0.3 | 52.2 | 43.9 |
| 288_1 | 0.06 | 83.9 | 73.2 |
| 288_1 | 0.3 | 44 | 43.5 |
| 289_1 | 0.06 | 99.9 | 93.3 |
| 289_1 | 0.3 | 52.5 | 51.5 |
| 290_1 | 0.06 | 129 | 132 |
| 290_1 | 0.3 | 108 | 104 |
| 291_1 | 0.06 | 83 | 72.7 |
| 291_1 | 0.3 | 84.9 | 86.3 |
| 292_1 | 0.06 | 124 | 119 |
| 292_1 | 0.3 | 98.1 | 93.3 |
| 293_1 | 0.06 | 161 | 170 |
| 293_1 | 0.3 | 74.2 | 68.2 |
| 294_1 | 0.06 | 113 | 117 |
| 294_1 | 0.3 | 126 | 129 |
| 295_1 | 0.06 | 74.5 | 67.9 |
| 295_1 | 0.3 | 105 | 102 |
| 296_1 | 0.06 | 96.9 | 91.4 |
| 296_1 | 0.3 | 71.7 | 72.3 |
| 297_1 | 0.06 | 129 | 122 |
| 297_1 | 0.3 | 84.8 | 81.8 |
| 298_1 | 0.06 | 170 | 177 |
| 298_1 | 0.3 | 103 | 108 |
| 299_1 | 0.06 | 61.8 | 54.2 |
| 299_1 | 0.3 | 41.9 | 38.2 |
| 300_1 | 0.06 | 95.6 | 81.9 |
| 300_1 | 0.3 | 64.5 | 57 |
| 301_1 | 0.06 | 79.7 | 59.5 |
| 301_1 | 0.3 | 25.1 | 16.9 |
| 302_1 | 0.06 | 172 | 136 |
| 302_1 | 0.3 | 102 | 104 |
| 303_1 | 0.06 | 169.5 | 56 |
| 303_1 | 0.3 | 23.6 | 22.9 |
| 304_1 | 0.06 | 86.2 | 79 |
| 304_1 | 0.3 | 80.1 | 84.7 |
| 305_1 | 0.06 | 121 | 112 |
| 305_1 | 0.3 | 84.1 | 77.5 |
| 306_1 | 0.06 | 85 | 83.3 |
| 306_1 | 0.3 | 76.5 | 75.2 |
| 307_1 | 0.06 | 85.3 | 75.7 |
| 307_1 | 0.3 | 42.5 | 35.8 |
| 308_1 | 0.06 | 80.1 | 66.8 |
| 308_1 | 0.3 | 63.2 | 51.6 |
| 309_1 | 0.06 | 105 | 87 |
| 309_1 | 0.3 | 71.2 | 56.6 |
| 310_1 | 0.06 | 93.1 | 90.9 |
| 310_1 | 0.3 | 75.2 | 72.2 |
| 311_1 | 0.06 | 73.9 | 64.9 |
| 311_1 | 0.3 | 108 | 111 |
| 312_1 | 0.06 | 108 | 93 |
| 312_1 | 0.3 | 73.9 | 78.4 |
| 313_1 | 0.06 | 114 | 107 |
| 313_1 | 0.3 | 88.9 | 78 |
| 314_1 | 0.06 | 118 | 115 |
| 314_1 | 0.3 | 83.4 | 76.9 |
| 315_1 | 0.06 | 128 | 120 |
| 315_1 | 0.3 | 87.8 | 92.1 |
| 316_1 | 0.06 | 117 | 128 |
| 316_1 | 0.3 | 115 | 132 |
| 317_1 | 0.06 | 81.9 | 77 |
| 317_1 | 0.3 | 116 | 115 |
| 318_1 | 0.06 | 108 | 93.2 |
| 318_1 | 0.3 | 108 | 96.6 |
| 319_1 | 0.06 | 127 | 124 |
| 319_1 | 0.3 | 71.8 | 68.4 |
| 320_1 | 0.06 | 55.5 | 50.1 |
| 320_1 | 0.3 | 81.5 | 86.6 |
| 321_1 | 0.06 | 131 | 123 |
| 321_1 | 0.3 | 93.8 | 89.3 |
| 322_1 | 0.06 | 92.8 | 90.8 |
| 322_1 | 0.3 | 75.5 | 82.5 |
| 323_1 | 0.06 | 60.7 | 56.5 |
| 323_1 | 0.3 | 76.9 | 71.5 |
| 324_1 | 0.06 | 89.4 | 75.8 |
| 324_1 | 0.3 | 67.2 | 63.5 |
| 325_1 | 0.06 | 75.7 | 66.8 |
| 325_1 | 0.3 | 64.4 | 53.6 |
| 326_1 | 0.06 | 90.4 | 85.1 |
| 326_1 | 0.3 | 83 | 79.4 |
| 327_1 | 0.06 | 84 | 76.3 |
| 327_1 | 0.3 | 92.8 | 99.5 |
| 328_1 | 0.06 | 77.3 | 76.4 |
| 328_1 | 0.3 | 61.6 | 67.3 |
| 329_1 | 0.06 | 136 | 137 |
| 329_1 | 0.3 | 106 | 90.2 |
| 330_1 | 0.06 | 196 | 193 |
| 330_1 | 0.3 | 111 | 115 |
| 331_1 | 0.06 | 108 | 112 |
| 331_1 | 0.3 | 86.9 | 82.1 |
| 332_1 | 0.06 | 123 | 126 |
| 332_1 | 0.3 | 126 | 135 |
| 333_1 | 0.06 | 108 | 106 |
| 333_1 | 0.3 | 91.5 | 91.2 |
| 334_1 | 0.06 | 138 | 137 |
| 334_1 | 0.3 | 83.5 | 187.7 |
| 335_1 | 0.06 | 96.5 | 87.7 |
| 335_1 | 0.3 | 90.5 | 75 |
| 336_1 | 0.06 | 153 | 144 |
| 336_1 | 0.3 | 103 | 86.6 |
| 337_1 | 0.06 | 71.8 | 62.8 |
| 337_1 | 0.3 | 77.9 | 66.3 |
| 338_1 | 0.06 | 81.4 | 68.4 |
| 338_1 | 0.3 | 117 | 110 |
| 339_1 | 0.06 | 73.5 | 54.9 |
| 339_1 | 0.3 | 133 | 124 |
| 340_1 | 0.06 | 53.2 | 44.4 |
| 340_1 | 0.3 | 27 | 22 |
| 341_1 | 0.06 | 116 | 115 |
| 341_1 | 0.3 | 72.5 | 65.9 |
| 342_1 | 0.06 | \|63.4 | 57.7 |
| 342_1 | 0.3 | 90.9 | 92.1 |
| 343_1 | 0.06 | \|62.6 | 67 |
| 343_1 | 0.3 | 57.9 | 58.9 |
| 344_1 | 0.06 | 104 | 99.5 |
| 344_1 | 0.3 | 65.2 | 56 |
| 345_1 | 0.06 | 112 | 123 |
| 345_1 | 0.3 | 79.8 | 77.9 |
| 346_1 | 0.06 | 64.2 | 62.5 |
| 346_1 | 0.3 | 26 | 20.8 |
| 347_1 | 0.06 | 82.8 | 84.2 |
| 347_1 | 0.3 | 53.3 | 53.8 |
| 348_1 | 0.06 | 124 | 134 |
| 348_1 | 0.3 | 71.5 | 71.3 |
| 349_1 | 0.06 | 102 | 104 |
| 349_1 | 0.3 | 89.8 | 90.3 |
| 350_1 | 0.06 | 122 | 103 |
| 350_1 | 0.3 | 79.3 | 70.3 |
| 351_1 | 0.06 | 137 | 136 |
| 351_1 | 0.3 | 129 | 130 |
| 352_1 | 0.06 | 106 | 91.7 |
| 352_1 | 0.3 | 90.6 | 79 |
| 353_1 | 0.06 | 102 | 97.2 |
| 353_1 | 0.3 | 83.3 | 83.2 |
| 354_1 | 0.06 | 85.6 | 73.4 |
| 354_1 | 0.3 | 97.8 | 90.8 |
| 355_1 | 0.06 | 62.8 | 55.3 |
| 355_1 | 0.3 | 18 | 15.1 |
| 356_1 | 0.06 | 123 | 113 |
| 356_1 | 0.3 | 124 | 122 |
| 357_1 | 0.06 | 89.7 | 76 |
| 357_1 | 0.3 | 72.1 | 63 |
| 358_1 | 0.06 | 78.4 | 64.9 |
| 358_1 | 0.3 | 73.5 | 75.7 |
| 359_1 | 0.06 | 105 | 103 |
| 359_1 | 0.3 | 86.2 | 82.1 |
| 360_1 | 0.06 | 121 | 114 |
| 360_1 | 0.3 | 105 | 97.3 |
| 361_1 | 0.06 | 98.5 | 101 |
| 361_1 | 0.3 | 73.4 | 67.2 |
| 362_1 | 0.06 | 152 | 145 |
| 362_1 | 0.3 | 136 | 132 |
| 363_1 | 0.06 | 121 | 129 |
| 363_1 | 0.3 | 90.2 | 88.1 |
| 364_1 | 0.06 | 105 | 122 |
| 364_1 | 0.3 | 123 | 122 |
| 365_1 | 0.06 | 88.2 | 96.4 |
| 365_1 | 0.3 | 62.1 | 54.9 |
| 366_1 | 0.06 | 99 | 89 |
| 366_1 | 0.3 | 72.5 | 72.7 |
| 367_1 | 0.06 | 94.9 | 94.5 |
| 367_1 | 0.3 | 102 | 106 |
| 368_1 | 0.06 | 102 | 92.1 |
| 368_1 | 0.3 | 128 | 107 |
| 369_1 | 0.06 | 121 | 140 |
| 369_1 | 0.3 | 94.1 | 105 |
| 370_1 | 0.06 | 85.5 | 77.2 |
| 370_1 | 0.3 | 154 | 176 |
| 371_1 | 0.06 | 118 | 102 |
| 371_1 | 0.3 | 115 | 101 |
| 372_1 | 0.06 | 94.9 | 93.3 |
| 372_1 | 0.3 | 62.6 | 58.4 |
| 373_1 | 0.06 | 118 | 108 |
| 373_1 | 0.3 | 95.1 | 98 |
| 374_1 | 0.06 | 97.1 | 106 |
| 374_1 | 0.3 | 99.4 | 102 |
| 375_1 | 0.06 | 91.4 | 86.9 |
| 375_1 | 0.3 | 73.5 | 69.7 |
| 376_1 | 0.06 | 126 | 109 |
| 376_1 | 0.3 | 97.9 | 93.4 |
| 377_1 | 0.06 | 89.1 | 77.2 |
| 377_1 | 0.3 | 77.2 | 66.7 |
| 378_1 | 0.06 | 76.1 | 64.2 |
| 378_1 | 0.3 | 82.1 | 68.2 |
| 379_1 | 0.06 | 89.6 | 81.4 |
| 379_1 | 0.3 | 75.4 | 74.5 |
| 380_1 | 0.06 | 115 | 116 |
| 380_1 | 0.3 | 90.5 | 99 |
| 381_1 | 0.06 | 121 | 107 |
| 381_1 | 0.3 | 86.6 | 94.2 |
| 382_1 | 0.06 | 95.5 | 96.9 |
| 382_1 | 0.3 | 84.3 | 91.5 |
| 383_1 | 0.06 | 95.5 | 97.9 |
| 383_1 | 0.3 | 70.6 | 69.1 |
| 384_1 | 0.06 | 142 | 145 |
| 384_1 | 0.3 | 212 | 220 |
| 385_1 | 0.06 | \|73.3 | 67.1 |
| 385_1 | 0.3 | 69.7 | 70.6 |
| 386_1 | 0.06 | 109 | 107 |
| 386_1 | 0.3 | 137 | 136 |
| 387_1 | 0.06 | 90 | 77.1 |
| 387_1 | 0.3 | 70.5 | 69.5 |
| 388_1 | 0.06 | 152 | 153 |
| 388_1 | 0.3 | 116 | 124 |
| 389_1 | 0.06 | 130 | 130 |
| 389_1 | 0.3 | 103 | 102 |
| 390_1 | 0.06 | 85.8 | 128 |
| 390_1 | 0.3 | 83.8 | 85.2 |
| 391_1 | 0.06 | 58.9 | 55.1 |
| 391_1 | 0.3 | 163.4 | 54.9 |
| 392_1 | 0.06 | 114 | 95.2 |
| 392_1 | 0.3 | 100 | 81.2 |
| 393_1 | 0.06 | 121 | 119 |
| 393_1 | 0.3 | 87.5 | 75.6 |
| 394_1 | 0.06 | 108 | 108 |
| 394_1 | 0.3 | \|59.8 | 58 |
| 395_1 | 0.3 | 87.4 | 87.7 |
| 396_1 | 0.06 | 83.7 | 75.5 |
| 396_1 | 0.3 | 80.2 | 70 |
| 397_1 | 0.06 | 195.7 | 94.4 |
| 397_1 | 0.3 | 78.3 | 71.8 |
| 398_1 | 0.06 | 101 | 87.2 |
| 398_1 | 0.3 | 91.2 | 82.9 |
| 399_1 | 0.06 | 105 | 88.7 |
| 399_1 | 0.3 | 109 | 99.7 |
| 400_1 | 0.06 | 154 | 155 |
| 400_1 | 0.3 | 96.4 | 105 |
| 401_1 | 0.06 | 90.3 | 86.5 |
| 401_1 | 0.3 | 100 | 86.3 |
| 402_1 | 0.06 | 81.2 | 72.8 |
| 402_1 | 0.3 | 36.6 | 31.2 |
| 403_1 | 0.06 | 114 | 109 |
| 403_1 | 0.3 | 118 | 114 |
| 404_1 | 0.06 | 74 | 58.9 |
| 404_1 | 0.3 | 69.7 | 63.7 |
| 405_1 | 0.06 | 106 | 103 |
| 405_1 | 0.3 | 111 | 112 |
| 406_1 | 0.06 | 92 | 72.5 |
| 406_1 | 0.3 | 60.9 | 53.6 |
| 407_1 | 0.06 | 103 | 102 |
| 407_1 | 0.3 | 113 | 105 |
| 408_1 | 0.06 | 83 | 80.2 |
| 408_1 | 0.3 | 86.5 | 77.6 |
| 409_1 | 0.06 | 117 | 119 |
| 409_1 | 0.3 | 61.1 | 53.2 |
| 410_1 | 0.06 | 107 | 95.8 |
| 410_1 | 0.3 | 86.8 | 88.9 |
| 411_1 | 0.06 | 80.9 | 81.1 |
| 411_1 | 0.3 | 56.4 | 52.6 |
| 412_1 | 0.06 | 68.8 | 63.1 |
| 412_1 | 0.3 | 66.5 | 60 |
| 413_1 | 0.06 | 105 | 93.7 |
| 413_1 | 0.3 | 85.5 | 82.1 |
| 414_1 | 0.06 | 110 | 111 |
| 414_1 | 0.3 | 73.9 | 73.2 |
| 415_1 | 0.06 | 94.7 | 93.3 |
| 415_1 | 0.3 | 68.8 | 62 |
| 416_1 | 0.06 | 104 | 90.2 |
| 416_1 | 0.3 | 64.6 | 58 |
| 417_1 | 0.06 | 95.3 | 85.2 |
| 417_1 | 0.3 | 64.7 | 54.8 |
| 418_1 | 0.06 | 69 | 61.3 |
| 418_1 | 0.3 | 68.9 | 60.8 |
| 419_1 | 0.06 | 105 | 107 |
| 419_1 | 0.3 | 167.9 | 66.8 |
| 420_1 | 0.06 | 115 | 92.4 |
| 420_1 | 0.3 | 64 | 57.1 |
| 421_1 | 0.06 | 100 | 104 |
| 421_1 | 0.3 | 77 | 74.7 |
| 422_1 | 0.06 | 105 | 98.4 |
| 422_1 | 0.3 | 68.2 | 56.2 |
| 423_1 | 0.06 | 73.8 | 60.5 |
| 423_1 | 0.3 | 66.2 | 63.2 |
| 424_1 | 0.06 | 84.1 | 72.7 |
| 424_1 | 0.3 | 73.1 | 72.9 |
| 425_1 | 0.06 | 96.8 | 93.6 |
| 425_1 | 0.3 | 72 | 73.8 |
| 426_1 | 0.06 | 120 | 109 |
| 426_1 | 0.3 | 96.5 | 85.2 |
| 427_1 | 0.06 | 102 | 104 |
| 427_1 | 0.3 | 79.7 | 72.8 |
| 428_1 | 0.06 | 88.9 | 77.2 |
| 428_1 | 0.3 | 53.5 | 49.8 |
| 429_1 | 0.06 | 41.2 | 34.4 |
| 429_1 | 0.3 | 17.3 | 13.2 |
| 430_1 | 0.06 | 72.7 | 64.2 |
| 430_1 | 0.3 | 48.2 | 41.6 |
| 431_1 | 0.06 | 61.7 | 53.2 |
| 431_1 | 0.3 | 28.1 | 23.7 |
| 432_1 | 0.06 | 61 | 64.7 |
| 432_1 | 0.3 | 26 | 26.1 |
| 433_1 | 0.06 | 42 | 32.4 |
| 433_1 | 0.3 | 26.1 | 23.7 |
| 434_1 | 0.06 | 49.6 | 41.6 |
| 434_1 | 0.3 | 13.7 | 10.8 |
| 435_1 | 0.06 | 102 | 105 |
| 435_1 | 0.3 | 134 | 159 |
| 436_1 | 0.06 | 135 | 145 |
| 436_1 | 0.3 | 93.3 | 94.9 |
| 437_1 | 0.06 | 79.7 | 74.6 |
| 437_1 | 0.3 | 44.9 | 42.5 |
| 438_1 | 0.06 | 85.8 | 79.5 |
| 438_1 | 0.3 | 79.2 | 75.9 |
| 439_1 | 0.06 | 71.6 | 72 |
| 439_1 | 0.3 | 80.6 | 74.6 |
| 440_1 | 0.06 | 96.1 | 00 |
| 440_1 | 0.3 | 97 | 94.4 |
| 441_1 | 0.06 | 98.6 | 84.8 |
| 441_1 | 0.3 | 171.9 | 63.2 |
| 442_1 | 0.06 | 137 | 125 |
| 442_1 | 0.3 | 81.8 | 84.2 |
| 443_1 | 0.06 | 117 | 106 |
| 443_1 | 0.3 | 67.3 | 58.9 |
| 444_1 | 0.06 | 70.8 | 58.6 |
| 444_1 | 0.3 | 38.4 | 34.8 |
| 445_1 | 0.06 | 115 | 123 |
| 445_1 | 0.3 | 83.5 | 82.1 |
| 446_1 | 0.06 | 99.8 | 88.6 |
| 446_1 | 0.3 | 77.2 | 70.8 |
| 447_1 | 0.06 | 92.7 | 95 |
| 447_1 | 0.3 | 52.7 | 55.1 |
| 448_1 | 0.06 | 59.7 | 48.1 |
| 448_1 | 0.3 | 75.3 | 65 |
| 449_1 | 0.06 | 75.2 | 76.2 |
| 449_1 | 0.3 | 24.6 | 25.2 |
| 450_1 | 0.06 | 91.1 | 77.4 |
| 450_1 | 0.3 | 68.8 | 68 |
| 451_1 | 0.06 | 117 | 121 |
| 451_1 | 0.3 | 37.6 | 34.4 |
| 452_1 | 0.06 | 74.4 | 74.6 |
| 452_1 | 0.3 | 43.3 | 39.5 |
| 453_1 | 0.06 | 67.4 | 60.2 |
| 453_1 | 0.3 | 55.9 | 53.3 |
| 454_1 | 0.06 | 73.9 | 63.4 |
| 454_1 | 0.3 | 30.6 | 22.8 |
| 455_1 | 0.06 | 48.4 | 40.7 |
| 455_1 | 0.3 | 20.7 | 17.1 |
| 456_1 | 0.06 | 72.5 | 57.7 |
| 456_1 | 0.3 | 54.8 | 44.3 |
| 457_1 | 0.06 | 134 | 125 |
| 457_1 | 0.3 | 72.1 | 73.9 |
| 458_1 | 0.06 | 107 | 98.7 |
| 458_1 | 0.3 | 66 | 56.2 |
| 459_1 | 0.06 | 87 | 72.9 |
| 459_1 | 0.3 | 87.9 | 83 |
| 460_1 | 0.06 | 106 | 102 |
| 460_1 | 0.3 | 79.8 | 75.8 |
| 461_1 | 0.06 | 91.5 | 76.5 |
| 461_1 | 0.3 | 95.3 | 88.6 |
| 462_1 | 0.06 | 102 | 88.5 |
| 462_1 | 0.3 | 75.7 | 69.7 |
| 463_1 | 0.06 | 74.1 | 71.3 |
| 463_1 | 0.3 | 64.8 | 64.6 |
| 464_1 | 0.06 | 109 | 98.8 |
| 464_1 | 0.3 | 56.2 | 51.8 |
| 465_1 | 0.06 | 83.8 | 79.2 |
| 465_1 | 0.3 | 65.5 | 64.2 |
| 466_1 | 0.06 | 120 | 108 |
| 466_1 | 0.3 | 78.9 | 92.1 |
| 467_1 | 0.06 | 97.2 | 91 |
| 467_1 | 0.3 | 87.6 | 84.6 |
| 468_1 | 0.06 | 101 | 92.6 |
| 468_1 | 0.3 | 100 | 104 |
| 469_1 | 0.06 | 158 | 152 |
| 469_1 | 0.3 | 93 | 90 |
| 470_1 | 0.06 | 103 | 102 |
| 470_1 | 0.3 | 61.3 | 56 |
| 471_1 | 0.06 | 151 | 131 |
| 471_1 | 0.3 | 105 | 102 |
| 472_1 | 0.06 | 108 | 107 |
| 472_1 | 0.3 | 118 | 114 |
| 473_1 | 0.06 | 126 | 126 |
| 473_1 | 0.3 | 120 | 114 |
| 474_1 | 0.06 | 88.2 | 76.5 |
| 474_1 | 0.3 | 62.2 | 50.1 |
| 475_1 | 0.06 | 125 | 97 |
| 475_1 | 0.3 | 94.8 | 91.8 |
| 476_1 | 0.06 | 111 | 121 |
| 476_1 | 0.3 | 89.3 | 89.3 |
| 477_1 | 0.06 | 93.7 | 94 |
| 477_1 | 0.3 | 90.2 | 89.3 |
| 478_1 | 0.06 | 84.2 | 87.6 |
| 478_1 | 0.3 | 84.2 | 88.7 |
| 479_1 | 0.06 | 91.1 | 84.3 |
| 479_1 | 0.3 | 99.6 | 100 |
| 480_1 | 0.06 | 82 | 83.9 |
| 480_1 | 0.3 | 69.5 | 59.7 |
| 481_1 | 0.06 | 95.3 | 86.8 |
| 481_1 | 0.3 | 60.3 | 48.9 |
| 482_1 | 0.06 | 96 | 100 |
| 482_1 | 0.3 | 102 | 102 |
| 483_1 | 0.06 | 78.7 | 73.8 |
| 483_1 | 0.3 | 43.8 | 36.4 |
| 484_1 | 0.06 | 80.1 | 72.5 |
| 484_1 | 0.3 | 70.1 | 64.2 |
| 485_1 | 0.06 | 86.2 | 83.4 |
| 485_1 | 0.3 | 46.7 | 41.3 |
| 486_1 | 0.06 | 101 | 96.1 |
| 486_1 | 0.3 | 59.9 | 59.1 |
| 487_1 | 0.06 | 111 | 112 |
| 487_1 | 0.3 | 88.3 | 87.3 |
| 488_1 | 0.06 | 125 | 124 |
| 488_1 | 0.3 | 113 | 111 |
| 489_1 | 0.06 | 114 | 105 |
| 489_1 | 0.3 | 88.2 | 87.2 |
| 490_1 | 0.06 | 89.8 | 85 |
| 490_1 | 0.3 | 50.1 | 45.1 |
| 491_1 | 0.06 | 117 | 104 |
| 491_1 | 0.3 | 57.3 | 55.3 |
| 492_1 | 0.06 | 88.1 | 76.2 |
| 492_1 | 0.3 | 68 | 65.1 |
| 493_1 | 0.06 | 61.8 | 51.2 |
| 493_1 | 0.3 | 52 | 47.1 |
| 494_1 | 0.06 | 153 | 135 |
| 494_1 | 0.3 | 109 | 101 |
| 495_1 | 0.06 | 103 | 108 |
| 495_1 | 0.3 | 106 | 104 |
| 496_1 | 0.06 | 127 | 122 |
| 496_1 | 0.3 | 82.3 | 79.5 |
| 497_1 | 0.06 | 66.8 | 60.2 |
| 497_1 | 0.3 | 66.6 | 66.5 |
| 498_1 | 0.06 | 97.7 | 95.1 |
| 498_1 | 0.3 | 84.9 | 75.7 |
| 499_1 | 0.06 | 94.2 | 87.9 |
| 499_1 | 0.3 | 73.4 | 69.6 |
| 500_1 | 0.06 | 147 | 129 |
| 500_1 | 0.3 | 79.3 | 73.6 |
| 501_1 | 0.06 | 106 | 99.9 |
| 501_1 | 0.3 | 71.8 | 64.3 |
| 502_1 | 0.06 | 67.9 | 66.1 |
| 502_1 | 0.3 | 53.3 | 52.3 |
| 503_1 | 0.06 | 123 | 121 |
| 503_1 | 0.3 | 88.4 | 84.7 |
| 504_1 | 0.06 | 85.7 | 72.8 |
| 504_1 | 0.3 | 41.4 | 36.7 |
| 505_1 | 0.06 | 190 | 180 |
| 505_1 | 0.3 | 117 | 119 |
| 506_1 | 0.06 | 124 | 113 |
| 506_1 | 0.3 | 116 | 102 |
| 507_1 | 0.06 | 90.7 | 84.9 |
| 507_1 | 0.3 | 94.8 | 102 |
| 508_1 | 0.06 | 73.2 | 73.6 |
| 508_1 | 0.3 | 49.4 | 48.1 |
| 509_1 | 0.06 | 74.7 | 73.4 |
| 509_1 | 0.3 | 50.7 | 41.7 |
| 510_1 | 0.06 | 83.7 | 66.8 |
| 510_1 | 0.3 | 28 | 23.9 |
| 511_1 | 0.06 | 71.5 | 72.7 |
| 511_1 | 0.3 | 43.6 | 41.1 |
| 512_1 | 0.06 | 54.7 | 48.2 |
| 512_1 | 10.3 | 30.1 | 23.8 |
| 513_1 | 0.06 | 96.9 | 98.2 |
| 513_1 | 0.3 | 45.4 | 41 |
| 514_1 | 0.06 | 70.5 | 63.9 |
| 514_1 | 0.3 | 21.6 | 16.9 |
| 515_1 | 0.06 | 65.5 | 55.1 |
| 515_1 | 0.3 | 27.1 | 23.5 |
| 516_1 | 0.06 | 107 | 82 |
| 516_1 | 0.3 | 68.7 | 59.9 |
| 517_1 | 0.06 | 49.5 | 38.6 |
| 517_1 | 0.3 | 18.6 | 12.8 |
| 518_1 | 0.06 | 33.1 | 25.6 |
| 518_1 | 0.3 | 9.6 | 5.9 |
| 519_1 | 0.06 | 35.9 | 31.6 |
| 519_1 | 0.3 | 10.2 | 7.3 |
| 520_1 | 0.06 | 29.6 | 22.4 |
| 520_1 | 0.3 | — | 7.1 |
| 521_1 | 0.06 | 84.8 | 80.2 |
| 521_1 | 0.3 | 24.1 | 17.7 |
| 522_1 | 0.06 | 88.6 | 89.8 |
| 522_1 | 0.3 | 41.4 | 38.4 |
| 523_1 | 0.06 | 45.2 | 34.4 |
| 523_1 | 0.3 | 14.8 | 11.6 |
| 524_1 | 0.06 | 65.2 | 61.4 |
| 524_1 | 0.3 | 32.1 | 27.3 |
| — | 0.06 | 103 | 95.2 |
| — | 0.3 | 66.5 | 60.9 |

From Table 8 it can be taken that the C4 pool is capable of reducing C4a mRNA and C4b mRNA efficiently at different concentrations.

1. (canceled)

2. (canceled)

3. (canceled)

4. (canceled)

5. (canceled)

6. (canceled)

7. (canceled)

8. (canceled)

9. A nucleic acid molecule of 12 to 30 nucleotides in length comprising a contiguous nucleotides sequence of at least 12 nucleotides which is at least 95% complementary to a mammalian C4 target sequence, wherein the nucleic acid molecule is capable of inhibiting the expression of a C4 mRNA.

10. The nucleic acid molecule according to claim 9, wherein the contiguous nucleotide sequence is fully complementary to a sequence selected from one or more of SEQ ID NOs: 3, 4, 6 and 7.

11. The nucleic acid molecule according to claim 9, wherein the nucleic acid molecule comprises a contiguous nucleotide sequence of 12 to 25.

12. The nucleic acid molecule of claim 9, wherein the nucleic acid molecule is a RNAi molecule.

13. The nucleic acid molecule of claim 9, wherein the nucleic acid molecule is a single stranded antisense oligonucleotide.

14. (canceled)

15. The nucleic acid molecule according to claim 9, wherein the nucleic acid molecule comprises one or more 2′ sugar modified nucleosides.

16. The nucleic acid molecule according to claim 15, wherein the one or more 2′ sugar modified nucleosides are independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

17. (canceled)

18. The nucleic acid molecule according to claim 9, where the contiguous nucleotide sequence comprises at least one phosphorothioate internucleoside linkage.

19. The nucleic acid molecule according to claim 18, wherein at least 90% of the internucleoside linkages within the contiguous nucleotide sequence are phosphorothioate internucleoside linkages.

20. The nucleic acid molecule according to claim 9, wherein the nucleic acid molecule, or contiguous nucleotide sequence thereof, comprises a gapmer of formula 5′-F-G-F′-3′, wherein regions F and F′ independently comprise 1-4 2′ sugar modified nucleosides and G is a region between 6 and 18 nucleosides which are capable of recruiting RNase H.

21. A pharmaceutically acceptable salt of a nucleic acid molecule according to claim 9.

22. A pharmaceutical composition comprising a nucleic acid molecule according to claim 9, or a pharmaceutically acceptable salt of the nucleic acid molecule and a pharmaceutically acceptable excipient.

23. An in vivo or in vitro method for inhibiting C4 expression in a target cell which is expressing C4, said method comprising administering a nucleic acid molecule according to claim 9, a pharmaceutically acceptable salt of the nucleic acid molecule, or a pharmaceutical composition comprising the nucleic acid molecule in an effective amount to said cell.

24. A method for treating a disease comprising administering a therapeutically or prophylactically effective amount of a nucleic acid molecule according to claim 9, a pharmaceutically acceptable salt of the nucleic acid molecule, or a pharmaceutical composition comprising the nucleic acid molecule, to a subject suffering from or susceptible to a neurological disease.

25. A method according to claim 24, wherein the neurological disease is selected from the group consisting of a tauopathy and schizophrenia.

26. (canceled)

27. (canceled)

28. (canceled)

29. (canceled)

30. (canceled)

31. A method for diagnosing a neurological disease in a patient suspected of a having a neurological disease, said method comprising the steps of
a) determining the amount of one or more C4 nucleic acids in a sample from the subject, wherein the determination comprises contacting the sample with one or more nucleic acid molecules as defined in claim 9,
b) comparing the amount determined in step a) to a reference amount, and
c) diagnosing whether the subject suffers from the neurological disease, or not, based on the results of step b).

32. The method of claim 31, wherein the sample is contacted in step a) with said one or more nucleic acid molecules under conditions which allow for the hybridization of said one or more nucleic acid molecules to said one or more C4 nucleic acids present in the sample, thereby forming duplexes of said nucleic acid molecules and said C4 nucleic acids.

33. A method for manufacturing a nucleic acid molecule as defined in claim 9, comprising reacting nucleotide units and thereby forming covalently linked contiguous nucleotide units comprised in the nucleic acid molecule.

34. The method of claim 33, wherein the method comprises the introduction of one or more sugar-modified nucleosides, of one or more modified internucleoside linkages, and/or of one or more modified nucleobases into the nucleic acid molecule.
